(19) United States
(12) Patent Application Publication
(10) Pub. No.: US 2024/0412851 A1
(43) Pub. Date: Dec. 12, 2024

(54) Method for training Machine learning system, method for generating resulting microscope image with a machine learning system, computer program product, and image processing system

(71) Applicant: Carl Zeiss Microscopy GmbH, Jena (DE)

(72) Inventors: Manuel Amthor, Jena (DE); Daniel Haase, Jena (DE); Volker Doering, Jena (DE); Markus Neumann, Jena (DE)

(21) Appl. No.: 18/732,764

(22) Filed: Jun. 4, 2024

(30) Foreign Application Priority Data
Jun. 7, 2023 (DE) 10 2023 115 087.1

(51) Int. Cl. G16H 30/40; G06T 5/50; G06V 20/70; G16H 30/20

(52) U.S. Cl. CPC G16H 30/40; G06T 5/50; G06V 20/70; G16H 30/20; G06T 2207/10056; G06T 2207/20084; G06T 2207/20221; G06V 2201/07

(57) ABSTRACT

A method for training a machine learning system having a processing model for a sample type, which processes microscope images of samples of the sample type by virtual processing mapping, comprising recording at least one fine stack of a sample of the sample type, wherein the at least one fine stack comprises microscope images of the sample registered with respect to one another, determining at least one target microscope image based on the fine stack and the virtual processing mapping, creating an annotated data set comprising at least the target microscope image and a learning microscope image, wherein the learning microscope image is based on a coarse stack capturing the sample coarser than the fine stack, optimizing the processing model on the basis of the annotated data set.

### RELATED APPLICATIONS

This application claims priority to German Patent Application No. 10 2023 115 087.1, filed on Jun. 7, 2023, which is incorporated herein by reference in its entirety.

### BACKGROUND OF THE INVENTION

In “Learning to Deblur”, Christian J. Schuler, Michael Hirsch, Stefan Harmeling and Bernhard Schölkopf describe a machine learning-based approach for blind deconvolution of images. For blind deconvolution, use is made of a neural network which has a deep layer architecture. For training, images from the ImageNet data set are artificially blurred, and the neural network is trained to refocus the blurred images. The authors show that, through a clever selection of categories from the ImageNet data set, images corresponding to their image category, for example images of valleys and tables, can be better trained on the basis of correspondingly selected images of the ImageNet data set. For successful training of the neural network, a large number of images are to be kept available.

In “Deep learning optical-sectioning method”, Xiaoyu Zhang et. al. describes a method which requires a pair of contrast images in order to enable the reconstruction of optically sectioned images. The pair of contrast images comprises a wide-field microscope image and an optically sectioned microscope image corresponding to the wide-field microscope image. For creating the optically sectioned microscope image, a sample is exposed multiple times by means of a method for structured illumination by means of a microscope, the corresponding optically sectioned image is reconstructed from the plurality of images. The method requires an arrangement for structured illumination for carrying out the method.

Furthermore, further methods for carrying out optical sectionings are known from the prior art. For example, systems are known in which fluorescent samples are illuminated with a known grating structure. The known grating structure is displaced and positioned on the sample in different orientations, and an optical sectioning can be calculated from the images recorded therewith.

Further optical sectioning methods comprise, for example, laser scanning microscopy, in which a sample is scanned point by point with a laser beam. As an alternative to a laser scanning microscope, so-called spinning aperture disks are also used in confocal microscopes in order to carry out optical sectionings.

The optical sectioning methods have in common that they increase the complexity of the microscopy system and thus the costs for the acquisition of a microscopy system.

According to an alternative method, a stack of a plurality of microscope images offset in height can be recorded by means of a wide-field microscope. An optically sharp sectioning can then be calculated from the stack of microscope images by means of a sample-independent deconvolution mapping. This so-called classic deconvolution mapping necessarily requires a plurality of microscope images offset in height and thus quite considerably increases the sample loading as a result of the recording of the stack.

Methods in which stacks of microscope images offset in height are initially recorded in order to generate either an image with super resolution, a denoised microscope image or a spectrally demixed microscope image therefrom are also known in the creation of super resolution images and in the denoising and spectral demixing of microscope images. These methods also thus considerably increase the loading of the sample.

### SUMMARY OF THE INVENTION

In order to prevent destruction of the samples, a robust and cost-effective method for training processing models for different processing mappings of microscope images, for example the determination of optical sectionings for optical sectioning applications, for the generation of microscope images with super resolution, the denoising and the spectral demixing in microscope images is required.

The invention relates to a method for training a machine learning system having a processing model for a sample type, comprising recording at least one learning stack, calculating a target microscope image for a learning microscope image, using the learning microscope image and the target microscope image as annotated data set and optimizing the processing model on the basis of the annotated data set, a method for generating a resulting microscope image with a machine learning system having a processing model for a sample type. The invention further relates to a computer program product and a computer-readable storage medium and an image processing system.

The invention is based on the object of providing a method which enables a simple and reliable training of a machine learning system having a processing model for a sample type, which processes microscope images from a sample of the sample type by means of a virtual processing mapping, in a cost-effective and reliable manner. In addition, the invention achieves the object of providing a system, a computer program and a computer-readable storage medium and an image processing system which generates resulting microscope images in a cost-effective and reliable manner.

One or more objects are achieved by the subject matters of the independent claims. Advantageous developments and preferred embodiments form the subject matter of the dependent claims.

An aspect of the invention relates to a method for training a machine learning system having a processing model for a sample type, which processes microscope images of samples of the sample type by means of virtual processing mapping, comprising: recording at least one fine stack, wherein the at least one fine stack comprises microscope images of a sample of the sample type, the microscope images registered to one another, determining at least one target microscope image based on the fine stack and the virtual processing mapping, preparing an annotated data set comprising at least the target microscope image and a learning microscope image, wherein the learning microscope image is based on a coarse stack capturing the sample coarser than the fine stack and in particular has more image artefacts than the target microscope image, and optimizing the processing model for learning the virtual processing mapping on the basis of the annotated data set of the sample type.

Samples can be any objects, fluids or structures. Each sample is suitably arranged and fixed in the optical path of a microscope by means of a sample carrier.

In the sense of the present invention, microscope images are all images recorded with a microscope, in addition the microscope images also comprise processed microscope images, in particular microscope images processed with the processing model.

In the prior art, extensive data sets of corresponding microscope images are often required for training processing models in order to train corresponding processing models. Alternatively, in the prior art, a microscope is expanded by further hardware components in order to expand the functional scope of the microscope accordingly.

The inventors of the present invention have recognized that, owing to the constantly increasing number of different sample types which are processed by users of microscopes, and because the different sample types have very different optical properties owing to the structures contained in the samples, it is not possible to provide a pre-trained processing model for each type of sample type and each possible virtual processing mapping to be applied to the sample, which provides satisfactory results in the processing of microscope images of the respective sample type for any sample or any sample type.

Therefore, the inventors propose an automated method, by means of which a corresponding processing model can be trained by recording at least one fine stack for executing a virtual processing mapping, wherein the virtual processing mapping corresponds to a classic processing mapping, for example, or the virtual processing mapping comprises an artifact removal mapping, by means of which a classic processing mapping can be improved. For this purpose, a target microscope image is determined from the fine stack based on the virtual processing mapping to be trained. If the classic processing mapping is a deconvolution, for example, and the processing model is to be trained for carrying out the deconvolution, then the target microscope image is a high-quality optical sectioning.

If a sufficient number of fine stacks are recorded by the sample and target microscope images are determined, coarse stacks corresponding to the fine stacks are recorded or determined. The coarse stack is designed such that it captures the sample coarser than the fine stack, for which reason the recording of the coarse stack, for example, loads the sample less or takes less time. Learning microscope images are respectively determined based on the coarse stacks. The learning microscope images and the target microscope images then form the annotated data set, for example. A processing model for executing the processing mapping is trained on the basis of the annotated data set.

The fully trained processing model can then calculate resulting microscope images based on coarse stacks, wherein a quality of the resulting microscope image determined from the coarse stack using the learned processing mapping using the processing model is comparable to the quality of the target microscope image calculated using the classic processing mapping based on the fine stack.

The present invention thus makes it possible to provide a method for training a processing model for a corresponding processing mapping for a multiplicity of user-specific samples or a wide variety of sample types, with the result that the user is able to train himself a corresponding processing model for each new sample type which the user uses or prepares, without additional costs arising for additional equipment or the creation or purchasing of new processing models. By means of the trained, sample-specific processing models, it is furthermore possible to use the respective sample-specific trained processing model also in the course of time again for samples of the same sample type for carrying out the learned virtual processing image, wherein the recording of coarse stacks is sufficient when using the processing model for evaluating recorded microscope images, for which reason the sample is preserved or the respective processing image is considerably improved by using the processing model.

The present invention can distinguish different sample types. Different sample types differ in that they have different optical properties in relation to the learned virtual processing mapping, and therefore a processing model trained by means of a first sample of a first sample type for carrying out a processing mapping cannot successfully process microscope images of a second sample of a second, different sample type.

Therefore, according to the present invention, a respective processing model is often trained for each sample type. The different sample types can be determined, for example, on the basis of context information of the respective sample. The context information can be stored, for example, in an image header of microscope images. Corresponding context information is also stored, for example, for the respective processing models trained for the sample types. According to the given context information, it can be determined whether a suitable, trained processing model is present, or whether a new sample type is available and a new processing model has to be trained.

In particular, the sample type can be determined on the basis of a first recording of a microscope image of a sample type or on the basis of an overview image recorded with an overview camera. In particular, the sample type can also be determined on the basis of the overview image and at least one microscope image of the sample type.

In particular, the sample type can be determined on the basis of context information.

In particular, the sample type is determined by a sample type determination model. The sample type determination model determines the sample type on the basis of one or more of the overview image, at least one microscope image or the context information.

The context information can in particular comprise one or more of:
a type of the sample which is imaged in the microscope images, that is to say information whether they are, for example, living cells, 3D cell cultures, developing organisms, oocytes or expanded samples, and also the sample depth and, if appropriate, the spatial extent of the sample, but furthermore samples from materials sciences or from the investigation of industrial materials are also conceivable,
a type of sample carrier which was used for recording the sample image, for example whether a chamber slide, a microtitre plate, a slide with cover glass or a Petri dish was used,
a manner in which the sample was prepared, this also includes, for example, a medium in which, for example, cells are present and a cell density resulting from the preparation,
image recording parameters, such as, for example, information about illuminance, illumination or detection wavelength(s), camera sensitivity, exposure time, filter settings, fluorescence excitation, contrast method, or sample table settings,
information about a microscope used, in particular about a microscope type used or a type of microscope used,
information about objects contained in the respective microscope image,
application information which indicates for which type of application the microscope images were recorded,
information about a user who has recorded the images.

A stack of microscope images can comprise at least one microscope image. If the stack comprises more than one microscope image, the microscope images are registered to one another, i.e. identical points in the sample are mapped onto identical image points in the microscope image.

During the optimizing of the processing model, also referred to as training of the processing model, model parameters of the processing model are defined on the basis of the training data. According to this invention, supervised learning is carried out. During the supervised learning, the annotated data set comprises at least the learning microscope image, also referred to as input microscope image, and the target microscope image. The learning microscope image is input into the processing model, the processing model calculates an output microscope image. An objective function captures a difference between the output microscope image, also referred to as resulting microscope image, of the processing model and a target microscope image of the annotated data set. The processing model is optimized by means of the objective function, i.e. parameters of the processing model are adapted in the optimization such that the objective function is optimized. The objective function is, for example, a loss function which is minimized.

According to some embodiments of the present invention, the supervised learning is, in particular, self-supervised learning, i.e. the method for training a machine learning system according to the present invention is, in particular, a method for self-supervised learning.

Self-supervised learning is understood to mean a special form of supervised learning, wherein the training data, in particular the annotated data set, are not provided manually, for example by manual annotations, but are automatically obtained or recorded and provided.

According to the present invention, the training data, i.e. the annotated data set, can be provided automatically, in particular by recording the fine stack, the coarse stack and by determining the target microscope image and the learning microscope image.

The loss function can capture, for example, differences between an image calculated by the processing model, the resulting microscope image, and a predefined target image, the target microscope image, pixel by pixel. The pixel by pixel differences can be added in absolute value (as absolute values) in an L1 loss function. The square sum of the pixel by pixel differences is formed in an L2 loss function. To minimize the loss function, the values of model parameters of the processing model are changed, which can be calculated, for example, by gradient descent and back propagation.

Instead of a loss function, the objective function can also comprise a gain function which is maximized.

Alternatively, other metrics can also be used to calculate the objective function, for example an L1 standard, an entropy loss, cross-entropy loss, hinge loss, dice loss, contrastive loss, consine loss, depending on the application, also by means of a Kullback-Leibler divergence, Wasserstein metric, minimax loss or earth mover distance or other known loss or gain functions.

Preferably, the coarse stack comprises only the learning microscope image and no further microscope images. Due to the fact that the coarse stack comprises only one learning microscope image, the annotated data set is particularly small, and consumes little memory space. However, since the virtual processing mapping was trained then with only one microscope image, it is clear that only one microscope image is also required in the inference, for which reason the viewing of a sample proceeds particularly gently since only one recording has to be taken.

According to the present invention, a coarse stack can be used during the training of a processing model; a coarse stack used in the training is also referred to as training coarse stack. Furthermore, according to the present invention, the processing model can be designed such that it processes coarse stacks. A coarse stack used in the inference is also referred to as inference coarse stack. A training coarse stack and an inference coarse stack resolve the mapped sample respectively coarser than a fine stack, in particular the training coarse stack and the inference coarse stack resolve the sample approximately identically, in particular the coarse stacks have the same granularity in the training and in the inference.

A sample is captured by recording image stacks, here for example a fine stack and a coarse stack. An image stack can capture a sample with different quality, wherein an image or an image stack with a higher quality captures a sample finer than an image or image stack with a poorer quality. Synonymously, the word granularity is also used in this application. A coarse or fine capture, i.e. the quality or also granularity with which an image stack captures a sample, can be in particular an axial or a lateral resolution. Likewise, however, the quality can also be a spectral resolution, wherein the spectral resolution can relate in particular to a spectral resolution in the recording of the microscope images and also in the illumination of the microscope images. In particular, the axial resolution can be a distance of neighboring microscope images in a z-stack. In addition, the quality or granularity of a stack can be given by the signal-to-noise ratio, for example in the case of a recording by means of a structured illumination, the quality of a super resolution mapping determined therefrom depends in particular on the signal-to-noise ratio. If, for example, shorter exposure times are used with a constant illumination, the signal-to-noise ratio decreases. In the sense of the present invention, an image with a poorer signal-to-noise ratio captures coarser than an image with a better signal-to-noise ratio.

In the sense of the present invention, stacks, also image stacks, can be any image stacks, for example z-stacks, lambda stacks, stacks of microscope images recorded using structured illumination patterns, wherein an orientation of structure used in the structured illumination varies over the stacks of the microscope images, or stacks of a plurality of noisy but otherwise identical microscope images. The microscope images in the image stacks are preferably registered to one another.

If, for example, the distance between two microscope images in a z-stack is increased, the z-stack with the greater distance captures the sample coarser than a z-stack with a smaller distance between the microscope images.

The processing model is preferably designed such that it can process image stacks and, for example, outputs processed image stacks. For this case, the objective function correspondingly captures the differences between the microscope images of the processed image stack and the microscope images of a target image stack.

The determining of the at least one target microscope image preferably comprises calculating the at least one target microscope image for at least one learning microscope image, wherein the learning microscope image is one of the microscope images of the fine stack or of the coarse stack and the coarse stack comprises at least the learning microscope image.

The method preferably also comprises a step of recording the coarse stack.

By virtue of the fact that the coarse stack is recorded separately from the fine stack, a particularly suitable capturing of the sample, for example selected depths in a sample, selected particularly well-suited spectral ranges or the like, can be carried out, for example.

The recording of the at least one fine stack is preferably carried out at a specific location of the sample, in particular the specific location is not needed for recording further microscope images of the sample, in particular the specific location of the sample is automatically selected by the machine learning system, for example in a predetermined region of the sample.

The recording of fine stacks is preferably carried out at in particular regular intervals during an experiment.

In particular, the recording of the fine stack is always carried out after a predefined number of recordings of coarse stacks, or for example always when a temporally variable sample has changed to such an extent that the coarse stacks can no longer be processed well. In particular, for this purpose, an output of the processing model is suitably evaluated after the recording of a coarse stack.

Temporally variable samples are also recorded in a multiplicity of experiments. In the case of temporally variable samples, in particular a renewed training of the processing model may be necessary since the sample has changed to such an extent that a proper evaluation is no longer possible. By means of a regular recording of fine stacks in the course of an experiment, it is thus possible to ensure that a fine stack is always also available for the renewed training of the processing model.

The recording of the fine stack is preferably carried out at the end of an experiment.

By virtue of the fact that the fine stack is recorded at the end of an experiment, an unnecessary loading of a sample is avoided during an experiment.

It is preferably possible to verify for each recorded coarse stack whether a suitable processing model for processing has been trained for the respective coarse stack or a corresponding fine stack has been recorded. If this is not the case, a corresponding fine stack is recorded at the same point in the sample.

In the case of rapidly varying samples, it may be necessary that the sample has already changed to such an extent after a few recordings of a coarse stack in the course of an experiment that the coarse stack can no longer be processed sufficiently well. It is therefore advantageous to verify, immediately after the recording of a coarse stack, whether the latter can also be processed or to be precise whether the processing with a processing model yields a result with a sufficient quality.

By virtue of the fact that a fine stack of a sample of the sample type is recorded at a specific point of the sample, the present invention makes it possible for a sample which is still to be imaged multiple times in the course of an experiment not to be excessively illuminated at a point or to be damaged by excessive illumination. For example, in specific samples, such as, for example, in the case of samples which are prepared and examined in Petri dishes or in microtitre plates, there are edge effects at the edge of the sample, which is why an examined cell structure or cell culture at the start of an experiment also provides good images, that is to say reasonable results, at the edge of the sample, but in the course of the experiment, for example, drying out can occur at the edge, which is why the cell structures at the edge die and the sample can no longer be used for recording microscope images of the cell structure after a specific time at the edge. If, however, the fine stack is recorded at the start of the experiment, then the cell structure at the edge of the sample is possibly damaged to such an extent by the recording of the fine stack that it can no longer be used in the later course of the experiment. Since, however, the cell structure is located in the edge region of the sample, this cell structure would not be usable for the later course of the experiment, since, according to experience, the sample dries out at the edge in the course of the experiment. That is to say, by means of the suitable selection of the specific location for recording the fine stack, the present invention makes it possible for regions of the sample which are intended to be repeatedly imaged or examined over a relatively long period of time not to be damaged by the recording of the fine stack, and therefore the number of cells which can be examined in the course of an experiment is kept high by means of a suitable selection of the specific location.

The specific location of the sample can preferably be selected by a user, preferably in an overview image of the sample. Contours of objects to be imaged in the sample are preferably pre-marked in the overview image.

A selection machine learning model can preferably be trained on the basis of the specific locations of the sample selected by a user to select the specific location of the sample independently. The selection machine learning model can be, for example, a pre-trained model for selecting specific locations, which is further trained on the basis of the user selection.

Before recording the fine stack, the method preferably comprises the steps of identifying one or more objects to be examined in the sample and controlling the machine learning system to record the fine stack on the basis of the identified objects to be examined in the sample.

Due to the fact that the objects to be examined in the sample are identified automatically, a user can quickly record a fine stack for his experiment and continue with the experiment accordingly.

An object identification model that has been trained to identify specific objects in samples is preferably used to identify objects to be examined. The use of the object identification model accelerates the identification of objects of interest.

The pre-trained processing model has preferably been trained on the basis of an in-domain data set. The training of the processing model can be significantly accelerated by in-domain pre-training.

The pre-trained processing model has preferably been trained on the basis of an out-of-domain data set.

For some sample types, there may be the situation that there are no in-domain data sets for pre-training. For such sample types, out-of-domain data sets can be used for pre-training; these too achieve an acceleration and improvement of the training of the processing model.

The processing model is preferably a stage processing model or an aggregate processing model. The stage processing model comprises a detail enhancement model and a decoupling model. The detail enhancement model is trained by means of the annotated data set to execute a detail enhancement mapping and the decoupling model classically calculates a decoupling mapping. The aggregate processing model is trained by means of the annotated data set to execute the virtual processing mapping comprising the detail enhancement mapping and the decoupling mapping or combining the detail enhancement mapping with the decoupling mapping. The annotated data set to train the stage model comprises either coarse stacks as learning microscope images and fine stacks as target microscope images or decoupling coarse stacks determined from the coarse stack by means of the decoupling mapping as learning microscope images and decoupling fine stacks determined from the fine stack by means of the decoupling mapping as target microscope images. The annotated data set to train the aggregate processing model comprises at least one microscope image of the coarse stack and at least one decoupled microscope image determined from the fine stack by means of the decoupling mapping as the target microscope image.

According to the present invention, the virtual processing mapping can be completely learned from an aggregate processing model. Alternatively, however, the virtual processing mapping can also be divided into different partial mappings, here the detail enhancement mapping and the decoupling mapping, the different partial mappings are then executed by partial models. For example, the decoupling mappings can comprise a classic processing mapping, for example a classic image processing mapping such as a deconvolution, a denoising, a spectral demixing or also a super resolution mapping. These classic processing mappings do not have to be learned with supervised training, but rather output a result without training. In these classic image processing mappings, it is important that input microscope images have a certain quality. By dividing the virtual processing mapping into a decoupling mapping and a detail enhancement mapping, the quality of the microscope images input into the decoupling mapping can be improved by means of the detail enhancement mapping, so that the decoupling mapping can output decoupled microscope images with a good quality.

If, on the other hand, an aggregate processing model is used, then the training is admittedly somewhat more complex, since a more complex processing mapping has to be trained, but the processing with the aggregate processing model is more efficient than with the stage processing model, since classic processing mappings are typically very computationally complex.

According to the present invention, a detail enhancement mapping is an image-to-image mapping of at least one input image to a result image learned in a supervised manner by a processing model, in particular a neural network, wherein the quality of the result image is better than the quality of the input image. For example, the input image has a poorer signal-to-noise ratio, a poorer spectral resolution or a poorer axial or lateral resolution. In particular, the detail enhancement mapping can be executed before or after the decoupling mapping. If the detail enhancement mapping is executed before the decoupling mapping, then the microscope images of a coarse stack form the input images and the resulting images output by the detail enhancement mapping have a quality like the microscope images of the fine stack, i.e. they capture the sample approximately as finely as a fine stack.

Preferably, a type of the virtual processing mapping is independent of the sample and the sample type, in particular the type of the virtual processing mapping is one or more of a deconvolution mapping, a super resolution mapping, a spectral demixing mapping, an artifact removal mapping or a denoising mapping.

Since the type of the processing mapping is independent of the sample or the sample type, an annotated data set can be created in a simple manner for each experiment by means of the processing mapping corresponding to the respective type of the virtual processing mapping, which annotated data set is then used in the training of the processing model for optimizing the processing model. This type of training of a machine learning system in which an annotated data set is created automatically is also referred to as self-supervised learning.

Preferably, the decoupling mapping is a deconvolution mapping, the microscope images of the fine stack and the coarse stack are offset in height with respect to one another, a distance of the microscope images offset in height with respect to one another is smaller in the fine stack than in the coarse stack, and the coarse stack thus captures the sample coarser than the fine stack.

Preferably, the coarse stack is a strict subset of the fine stack.

If, for example, the fine stack comprises n microscope images, the coarse stack can in particular comprise only one to n−1 microscope images. Since the coarse stack comprises fewer microscope images than the fine stack, the coarse stack captures the sample coarser than the fine stack, since at least one of the microscope images of the fine stack is not part of the coarse stack.

Classic deconvolution mappings require a high computational effort. According to the present invention, a method for training a processing model for carrying out a deconvolution mapping can be provided; with the trained processing model, the computational effort when creating deconvoluted microscope images is quite considerably reduced.

Preferably, the distance of the microscope images offset in height along a height of the fine stack is selected, for example, depending on the sample type or on context information, in particular the method comprises determining the distance of the microscope images offset in height in the fine stack and/or in the coarse stack, wherein the distance is determined depending on the sample type or depending on the context information and in particular an imaging device such that the deconvolution mapping can be carried out, and an automatic recording of the fine stack is carried out on the basis of the determined distance.

An imaging device in the sense of the present invention is any combination of optical components by means of which an image of a sample can be generated. In particular, the imaging device can be a microscope.

Preferably, the distance of the microscope images offset in height is selected independently of the sample type and the imaging device. In particular, the distance of the microscope images offset in height is selected as desired.

Preferably, the processing mapping is, for example, a deconvolution mapping and the distance is selected according to the Shannon-Nyquist scanning theorem.

In order to be able to carry out a deconvolution mapping, the distances of the microscope images offset in height in the fine stack must be selected depending on the sample type, in particular depending on the imaging device. For example, on the basis of the structures to be examined according to the Shannon-Nyquist scanning theorem. This enables the deconvolution and thus an optical sectioning to be created with good optical quality, with the result that a correspondingly trained virtual processing mapping with samples of the same sample type can achieve a similar mapping quality.

If the virtual processing mapping or the decoupling mapping is a deconvolution mapping, the coarse stack is preferably a strict subset of the fine stack. For example, the coarse stack comprises every second, every third or every fourth of the microscope images of the fine stack. For example, the coarse stack comprises m of the n microscope images of the fine stack, wherein men and the m microscope images are selected as desired from the n.

As a result of the fact that the coarse stack is a strict subset of the fine stack, a sample loading can be further reduced since, during the training of the processing model, a coarse stack for the training of the processing model does not yet have to be recorded in addition to the fine stack.

Preferably, the distance of the microscope images offset in height of the coarse stack is selected precisely such that, in two neighboring microscope images of the coarse stack in the microscope images, objects which do not lie in the focus of the imaging device but are nevertheless reproduced in the microscope images, even if blurred, are not further away from one another than is required by the Shannon-Nyquist scanning theorem.

Preferably, a depth-variant point spread function is used in the deconvolution mapping.

As a result of the fact that a depth-variant point spread function is used in the deconvolution mapping, the deconvolution can also reliably carry out a deconvolution and thus the calculation of the optical sectioning at the edge of a sample and in the depth profile of a sample.

Preferably, the fine stack and the coarse stack comprise an image stack recorded using a light sheet microscope with microscope images offset in height.

Even if light sheet microscopes already mean a considerably lower light loading for the samples compared with, for example, wide-field microscopes, a light loading of a sample can be further reduced by the use of the processing mapping trained in a sample-type-specific manner.

Preferably, the decoupling mapping is a spectral demixing mapping, wherein the fine stack and the coarse stack are each lambda stacks, wherein the microscope images of a lambda stack each capture a different spectral range of a spectrum, in particular a continuous spectrum, the microscope images of the coarse stack capture the spectrum coarser than the microscope images of the fine stack, in particular the coarse stack comprises fewer microscope images than the fine stack, and/or in particular the coarse stack resolves the captured spectrum coarser than the fine stack.

Preferably, the capturing of the spectrum is adapted or varied by one or more of:
varying the excitation spectrum for excitation of fluorophores contained in the sample, in particular the excitation spectrum is a continuous spectrum and/or a discrete spectrum, and in particular the excitation spectrum is varied such that the different excitation spectra used capture the spectrum coarser or finer;
varying filters used in the beam path of an image capturing device between the capturing of the fine stack and the coarse stack, which filter the excitation spectrum and/or the fluorescence spectrum, in particular filters with different bandwidths can be used, in particular filters with narrower bandwidths are used during the capturing of the fine stack than during the capturing of the coarse stack, or fewer spectral ranges are captured during the capturing of the coarse stack than during the capturing of the fine stack; or
combining a plurality of microscope images of the fine stack to form a microscope image of the coarse stack.

Since the excitation spectrum is varied during the capturing of the coarse stack such that the spectrum is captured coarser, a light loading during the illumination of the sample can be minimized.

As a result of the fact that different filters are used during the capturing of the fine stack and the coarse stack, in particular filters with wider bandwidths are used during the capturing of the coarse stack, fewer recordings for the capturing of the coarse stack have to be recorded, as a result of which a light loading of the sample can be reduced.

As a result of the fact that images of the fine stack are simply combined to form images of the coarse stack for the generation of the coarse stack, a light loading of the sample can be further reduced since the coarse stack does not have to be recorded separately.

A spectrum during the examination of samples can be continuous or discrete. The spectrum can be divided into different spectral ranges, the division into spectral ranges can be coarser or finer, in that greater or smaller spectral ranges and correspondingly more or less different spectral ranges are selected. For example, the fine stack can be recorded with a first number of discrete spectra and the coarse stack with a second number of discrete spectra, wherein the second number is smaller than the first number. For example, the spectra can be line spectra, and only every second, third or, for example, fourth line is used for the illumination of the sample during the capturing of the coarse stack, and the sample is thus captured coarser during the capturing of the coarse stack.

A wide variety of fluorophores are used in fluorescence microscopy, often a plurality of fluorophores with overlapping absorption and emission spectra. In addition, autofluorescence of objects contained in the sample occurs in samples. Such autofluorescent objects are usually undesired objects or objects of no interest for the respective recording, which impair, in particular worsen, the visibility of the objects of actual interest in the sample. Classically, such autofluorescence objects can be removed with the aid of a spectral demixing mapping. In addition, samples in which the objects of interest are made visible by means of different fluorophores are often examined, which is why the objects are visible to different degrees using different illuminations and/or in different spectral ranges. In this case, the different excitation and emission spectra of the objects marked with different fluorophores often overlap or overlap, which is also referred to as bleed through. In order to separate the objects and thus the different excitation and emission spectra well from one another, so-called lambda stacks are recorded, on the basis of which classic spectral demixing methods are used. For the classic spectral demixing, a lambda stack which is as detailed and/or spectrally fine-resolved as possible is required. This is decomposed into its spectral components by means of the spectral demixing, wherein different fluorophores occurring in a sample and an autofluorescence component of the sample are each treated as separate spectral components or components and can thus be separated from one another. As a result of the fact that a spectral demixing mapping can be trained by means of the present invention, in which a spectrum only has to be recorded or imaged in a coarsely resolved manner, the present invention makes it possible to reduce the sample loading during the recording of a lambda stack to be used in the spectral demixing.

Preferably, the fine stack has a first number of microscope images, wherein each of the microscope images of the fine stack corresponds to a specific spectral range, and the coarse stack has a second number of microscope images.

Preferably, the decoupling mapping is a denoising mapping, wherein the fine stack comprises a plurality of noisy microscope images recorded with the same recording parameters, and the denoising mapping calculates a denoised target microscope image from the plurality of noisy microscope images in the fine stack, and a determining of the coarse stack comprises selecting a strict subset of the noisy microscope images of the fine stack as a coarse stack, i.e. if the fine stack in particular comprises n microscope images, the coarse stack then in particular comprises one to n−1 microscope images and thus captures the sample coarser than the fine stack.

According to an alternative, the coarse stack can also have been exposed with a shorter exposure time or a lower illumination intensity, for which reason a noise in the coarse stack is lower than in the fine stack. The learning microscope image is then an image in the coarse stack, and the target microscope image is then an image of the fine stack.

Usually, a noise level or a signal-to-noise ratio of a recording is reduced by increasing the exposure time or the brightness or the illumination intensity during the recording. As a result of the increased illumination intensity during the recording, however, the sample is additionally loaded and can be damaged. The present invention therefore trains a processing model to generate a denoised image from a few noisy images with an identical field of view. The present invention thus constitutes a method for training a sample-specific denoising mapping which makes it possible to minimize the sample loading during the noise reduction.

Preferably, the decoupling mapping is a super resolution mapping, and the recording of the fine stack and the coarse stack comprises illuminating the sample with a structured illumination pattern and changing the illumination pattern on the sample such that a phase position of the illumination pattern in the sample is different for different microscope images of a stack. In particular, an exposure time during the recording of the coarse stack is shorter than during the recording of the fine stack, or an illumination intensity during the recording of the coarse stack is lower than during the recording of the fine stack, such that the microscope images of the coarse stack have a lower signal-to-noise ratio than the microscope images of the fine stack, and the coarse stack thus captures the sample coarser than the fine stack.

By virtue of the fact that the processing model is trained for carrying out a super resolution mapping, a loading and an exposure time when creating super resolution mappings can be considerably reduced. Furthermore, the providing of the processing model thus also makes it possible for an exposure time to be considerably reduced during the super resolution mapping, as a result of which, in particular, the imaging of dynamic processes in samples, so-called life cell imaging, is considerably improved.

Preferably, the structured illumination pattern comprises one or more of a line grid, a point grid, a square point grid or a hexagonal point grid, and the varying of the structured illumination pattern comprises shifting the phase position and/or changing the orientation of the structured illumination pattern.

As a result of the fact that a wide variety of illumination patterns can be used in order to create the coarse stack or the fine stack, the training of the processing model is particularly flexible.

Preferably, the illuminating with the structured illumination pattern comprises mixing high-frequency components of the structured illumination pattern with high-frequency components of structures in the sample, wherein different mixed high-frequency components are formed by shifting the phase position of the illumination pattern respectively depending on the phase position of other high-frequency components of structures of the sample with other high-frequency components of the illumination pattern, and the different mixed high-frequency components are captured in different ones of the microscope images of a stack, and the calculating of the target microscope image comprises demixing the different mixed high-frequency components by means of the super resolution mapping in order to calculate the super resolution microscope image.

In particular, the high-frequency components of the illumination pattern and the high-frequency components of the structure comprise frequencies which cannot be captured by the imaging device due to the resolution capability of the imaging device, while the mixed high-frequency components are shifted in the frequency spectrum such that the imaging device can capture the mixed structures in the microscope images as structures with a lower frequency.

Preferably, the demixing comprises deconvolution using a point spread function, wherein the point spread function is a filtered point spread function by means of which certain ones of the mixed high-frequency components are filtered out.

By suitable combining and filtering, microscope images with an improved resolution, a so-called super resolution, can be created from microscope images which have been suitably recorded with structured illumination patterns. According to the present invention, a machine learning system can train a processing model by means of the automated method for carrying out the super resolution mapping for a certain sample type. The super resolution mapping can furthermore be further improved if a filtered point spread function is used, such filtered point spread functions filter out certain high-frequency components.

The determining of the at least one target microscope image preferably comprises calculating one or more decoupled candidate microscope images and in particular selecting the target microscope image from the plurality of decoupled candidate microscope images, wherein in the calculating of the plurality of decoupled candidate microscope images a different set of parameters of the deconvolution mapping is used for each of the plurality of decoupled candidate microscope images, wherein by means of the used parameters for example a used decoupling algorithm, a number of iterations of the used decoupling algorithm, used correction methods and correction parameters of the used correction method are selected.

By virtue of the fact that the target microscope image is selected from a plurality of decoupled candidate microscope images, it is possible to ensure that an optimally decoupled target microscope image is selected from the decoupled candidate microscope images depending on the sample type.

The determining of the at least one target microscope image preferably comprises verifying the at least one target microscope image which in particular determines whether the processing mapping has successfully processed the learning microscope image.

For example, a sharpness, for example an edge sharpness, of the target microscope image and of the learning microscope image can be determined. On the basis of the respective sharpness which results, for example, from the width of edges determined in the respective microscope images, the two images are compared and it is thus possible to determine whether the processing mapping or the decoupling mapping, for example the deconvolution, has been executed optimally. In a similar manner, the candidate microscope images can also be compared with one another in order to determine an optimal candidate image. By virtue of the fact that the calculation of the target microscope image is verified, it is possible to ensure that the deconvolution mapping functions reliably and correspondingly the annotated data set includes a target microscope image which is as good as possible and on the basis of which the processing model for executing the processing mapping or the detail enhancement mapping is trained.

The determining of the at least one target microscope image preferably comprises calculating a plurality of target microscope images.

Depending on a depth of the sample, it is possible for a fine stack to comprise so many microscope images that a plurality of target microscope images can be calculated from a fine stack. Therefore, the data volume in the annotated data set can be increased from the plurality of target microscope images together with the corresponding learning microscope images of the fine stack, which improves the training of the processing mapping. For example, in order to carry out a deconvolution mapping of a microscope image, two microscope images arranged above the microscope image and two microscope images arranged below the microscope image are required, for example. If, for example, fewer microscope images are used, the quality of the deconvolution decreases, the number is increased to in each case three, four or five microscope images arranged below and above the microscope image to be deconvoluted, and the quality of the deconvolution is correspondingly improved.

The annotated data set preferably comprises a plurality of target microscope images and one or more corresponding learning microscope images for each of the target microscope images.

Due to the annotated data set comprising a plurality of image pairs consisting of learning microscope image and target microscope image, a training of the deconvolution mapping and thus the quality of the optical sectioning obtained can be improved.

The processing model preferably comprises an encoder-decoder network, in particular a U-Net, isotropic network architectures, a generator of a generative adversary network or a transformer, in particular vision transformer models.

For example, the processing model comprises an isotropic network architecture, i.e. a network architecture which has no down- and up-sampling paths, but the resolution of the internal feature maps is kept constant.

For example, the processing model is or comprises a generator of a generative adversary network. A generative adversarial network (GAN) comprises at least one generator and one discriminator. In the present case, the generator receives the learning microscope images as input and generates resulting microscope images therefrom. The discriminator receives either the output of the generator or a target microscope image of the annotated data set as input data. The output of the discriminator is also called discrimination result. The discrimination result indicates whether the input image is a resulting microscope image output by the generator or a target microscope image of the annotated data set. The generator and the discriminator are trained jointly. In a joint objective function of the generator and of the discriminator, the outputs thereof are captured. In the objective function, the generator is penalized if the discriminator identifies output data output by the generator as such and the discriminator is penalized if it incorrectly classifies output data output by the generator as target microscope image. An objective function in the training of the GAN is in particular a minimax-loss or also a Wasserstein-loss.

The training of the processing model is preferably either a learning from scratch of the processing model or a transfer learning of a pre-trained processing model, in particular the pre-trained processing model is selected from a number of pre-trained processing models on the basis of the sample type.

In particular, the pre-trained processing model is selected on the basis of an experiment carried out, in particular on the basis of context information, in particular on the basis of one or more experiment settings, for example illumination source, (for example laser), wavelength used, exposure time, and objective used.

Since the training of the processing model can also be carried out on the basis of a pre-trained processing model, wherein the pre-trained processing model is selected, for example, on the basis of the sample type, the computational effort when training the processing model can be reduced, as can the duration of the training.

The annotated data set preferably comprises, in addition to a microscope image pair consisting of the target microscope image and the learning microscope image, further microscope image pairs, wherein the further microscope image pairs are calculated, for example, by data augmentation from an original microscope image pair or were calculated from the target microscope image by means of a simulation utilizing a point spread function of the deconvolution mapping, wherein the point spread function is, for example, a depth-variant point spread function.

An augmentation is understood to mean, for example, transformations which transform an input image such that the objects correspondingly contained in the microscope image can still be identified; here, for example, geometric augmentations such as rotations, translations, mirroring, scaling, spreading, stretching and compression, deforming by means of an elastic grid and the like are mentioned. Alternatively, however, optical augmentations such as gray value transformations, brightening, darkening (in each case additively or multiplicatively), adjusting a gamma correction value, vignetting, offsetting, color inversion, artificial noise, undersampling, down-sampling and histogram spreading can also be used. The augmentation can also be used such that only a part of the microscope image, for example a region of interest, is input into the processing model.

As a result of the fact that the annotated data set can be increased by the calculation of further pairs, the training can more reliably learn the virtual processing mapping.

A further aspect of the present invention relates to a method for generating a resulting microscope image with a machine learning system having a processing model for microscope images of samples of a sample type. The method comprises the steps of: providing a processing model for carrying out a virtual processing mapping for microscope images of the sample type, wherein a processing model is used which has been trained using a method for training a machine learning system as described above, recording a coarse stack to be processed comprising a plurality of microscope images of the sample of the sample type, calculating a resulting microscope image from the coarse stack by means of the virtual processing mapping, characterized in that the coarse stack to be processed resolves the sample coarser than the fine stack, in particular the number of microscope images in the coarse stack to be processed is smaller than the number of microscope images in the fine stack.

In conventional, classic optical processing mappings, either further hardware components are required in the recording microscope in order to obtain good optical sectionings, or the samples have to be considerably loaded by the production of 3D recordings, which in turn can lead to poorer data, for example up to the destruction of the sample.

Furthermore, machine learning-based methods for image improvement are also known from the prior art; these use in particular deep neural networks for image-to-image transformations. In principle, optical sectioning methods can also be carried out with such deep neural networks. However, such machine-learned models are often highly sample-dependent, for which reason it is not possible to provide central models for all possible user samples.

Conventional methods for generating a resulting microscope image either require expensive hardware for processing microscope images, for example for creating an optical sectioning, or the sample under consideration is exposed to such an extent during the creation of microscope image stacks that it is damaged during the illumination.

According to the present invention, the user can provide a processing model on his microscope himself according to a present sample type, which he himself has trained by means of a suitable recording of a fine stack on a sample of the sample type. By virtue of the provision of a trained processing model, the present invention thus enables classic optical processing mappings to be simulated in a processing model using recordings of a fine stack having microscope images of samples of a sample type, the microscope images registered to one another, and consequently to process the samples of the sample type by means of the virtual processing mapping learnt by the processing model without excessively loading the samples with a multiplicity of further complex recordings of further microscope image stacks and without a user having to upgrade an existing microscope with expensive hardware.

The method preferably further comprises, before the providing of the processing model, verifying whether a suitable processing model having a suitable processing mapping for the sample type is available and, if not, executing the method for training a machine learning system using the sample according to the method described above, in particular the fine stack being created in a predetermined region of the sample and in particular the coarse stack to be processed being recorded in a region of the sample different from the predetermined region.

As a result of the fact that, before the providing of the processing model, it is verified whether a suitable processing model for the sample type is available, for a sample of a specific sample type, if a virtual processing mapping has already been trained for this sample type, a new training can be dispensed with and instead recourse can be had to the learned processing model having the suitable processing mapping.

As a result of the fact that the fine stack was recorded at a different point than the coarse stack, for example at a predetermined point or a predetermined region in the sample, it is possible to prevent particularly interesting regions in the sample from being excessively loaded during the creation of the annotated data set or during the execution of the method for training a machine learning system.

The number of microscope images in the coarse stack to be processed is preferably equal to the number of microscope images in the coarse stack of the annotated data set, in particular the coarse stack to be processed and the coarse stack of the annotated data set comprising a plurality of microscope images.

As a result of the fact that the target microscope image is calculated from a plurality of input microscope images of the fine stack, the quality of the processing mapping can be improved. Furthermore, a target stack can be calculated.

For example, in the deconvolution mapping, the microscope images offset in height in the result stack can be based on a plurality of the microscope images offset in height of the coarse stack, for which reason the microscope images of the target stack can likewise have an improved quality.

Preferably, the recording of microscope images according to the methods described above comprises recording microscope images at different wavelengths, the microscope images at different wavelengths being processed independently of one another using the processing mapping or by means of the virtual processing mapping and the processed microscope images at different wavelengths subsequently being suitably assembled.

As a result of the fact that microscope images at different wavelengths are recorded and processed, the processed microscope images can be assembled again in a correspondingly supporting and weighted manner with respect to one another, as a result of which the quality of the processing mapping can be further improved.

A further aspect of the invention relates to a machine learning system comprising means for carrying out the methods described above.

A further aspect of the invention relates to a computer program comprising instructions which, when the program is executed by a computer, cause the latter to carry out the method described above.

A further aspect of the invention relates to a computer-readable storage medium comprising instructions which, when the instructions are executed by a computer, cause the latter to carry out the method described above.

A further aspect comprises an image processing system comprising an evaluation device, wherein the evaluation device comprises a processing model which has been trained according to the method described above, wherein the evaluation device is in particular designed to process the images recorded with the imaging device by means of the method described above.

All aspects and variations of the respective aspects described here have in common that a processing model can be trained which can output resulting microscope images based on further coarse stacks, the quality of which approximately corresponds to the quality of the target microscope images, in each case by the manner in which the fine stacks and the coarse stacks are recorded or determined and how the target microscope images are determined therefrom. All aspects and variations moreover have in common that the sample is loaded significantly less by the illumination during the recording of the coarse stacks than during the recording of the fine stack, therefore the disclosed method achieves a conservation of the sample during the examination, but without worsening the quality of the results. Furthermore, the process time during the recording is considerably reduced since fewer microscope images or microscope images with, for example, shortened exposure times are recorded, for which reason, for example, dynamic processes and the like can also be captured significantly better.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 schematically shows a system for use with the method for generating a resulting microscope image with a processing model according to one embodiment;

FIG. 2 schematically shows an evaluation device for generating a resulting microscope image with a processing model according to one embodiment;

FIG. 3 schematically shows a processing model with a plurality of layers according to one embodiment;

FIG. 4 is a schematic illustration of a method for generating a resulting microscope image according to a further embodiment;

FIG. 5 is a schematic illustration of processes of a method according to a further embodiment;

FIG. 6 is a schematic illustration for better understanding of a method according to one embodiment;

FIG. 7 is a schematic illustration for better understanding of a method according to a further embodiment.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

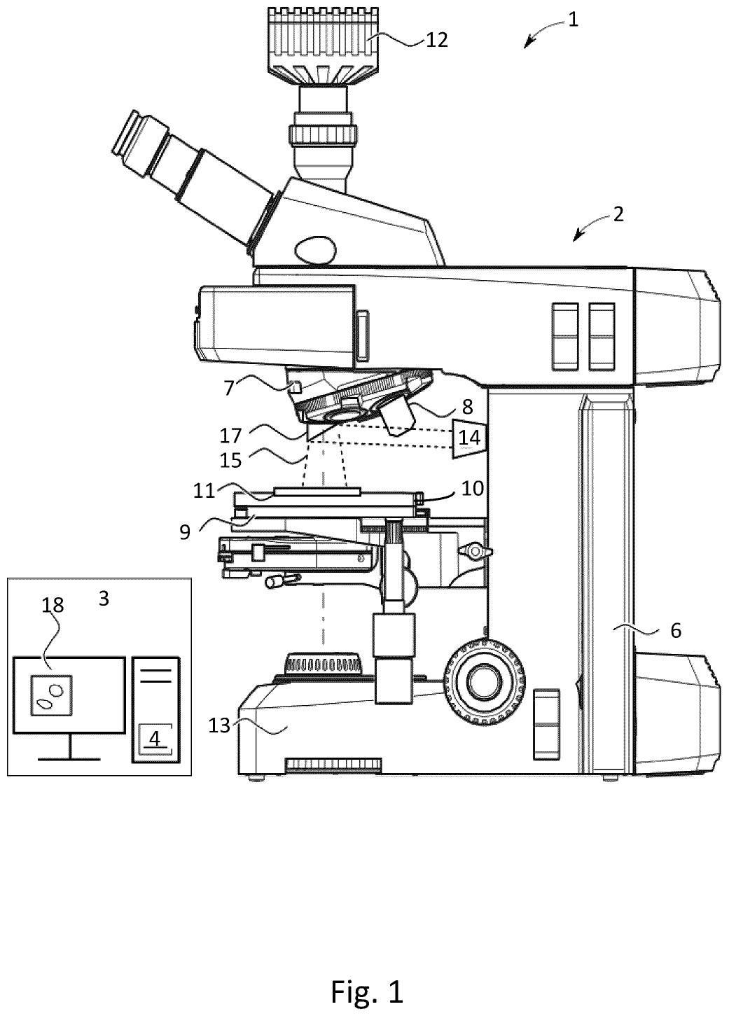

An exemplary embodiment of a machine learning system comprises a microscope 2, a control apparatus 3 and an evaluation device 4. The microscope 2 is communicatively coupled to the evaluation device 4. The evaluation device comprises a processing model 5. The processing model 5 can be trained with microscope images captured by the microscope 2 to carry out a virtual processing mapping (FIG. 1).

According to the first embodiment, the microscope 2 is a wide-field light microscope. The microscope 2 comprises a stand 6 which contains further microscope components. The further microscope components are in particular an objective changer or objective turret 7 with a mounted objective 8, a sample stage 9 with a holding frame 10 for holding a sample carrier 11 and a microscope camera 12. If a sample is clamped into the sample carrier 11 and the objective 8 is pivoted into the microscope optical path, and an illumination device 13 illuminates the sample, the microscope camera 12 receives detection light from the clamped sample and can record a microscope image. Samples can be any objects, fluids or structures.

The microscope 2 optionally comprises an overview camera 14 with which overview images of a sample environment can be recorded. The overview images show the sample carrier 11, for example. A field of view 15 of the overview camera 14 is larger than a field of view during a recording of a microscope image 16. The overview camera 14 looks at the sample carrier 11 by means of a mirror 17. The mirror 17 is arranged on the objective turret 7 and can be selected instead of the objective 8.

According to this embodiment, the control apparatus 3, as illustrated schematically in FIG. 1, comprises a screen 18 and the evaluation device 4. The control apparatus 3 is configured to control the microscope 2 to record microscope images 16 and to store microscope images 16 recorded by the microscope camera 12 on a memory module 19 of the evaluation device 4 and to display them on the screen 18. The recorded microscope images 16 are further processed by the evaluation device 4.

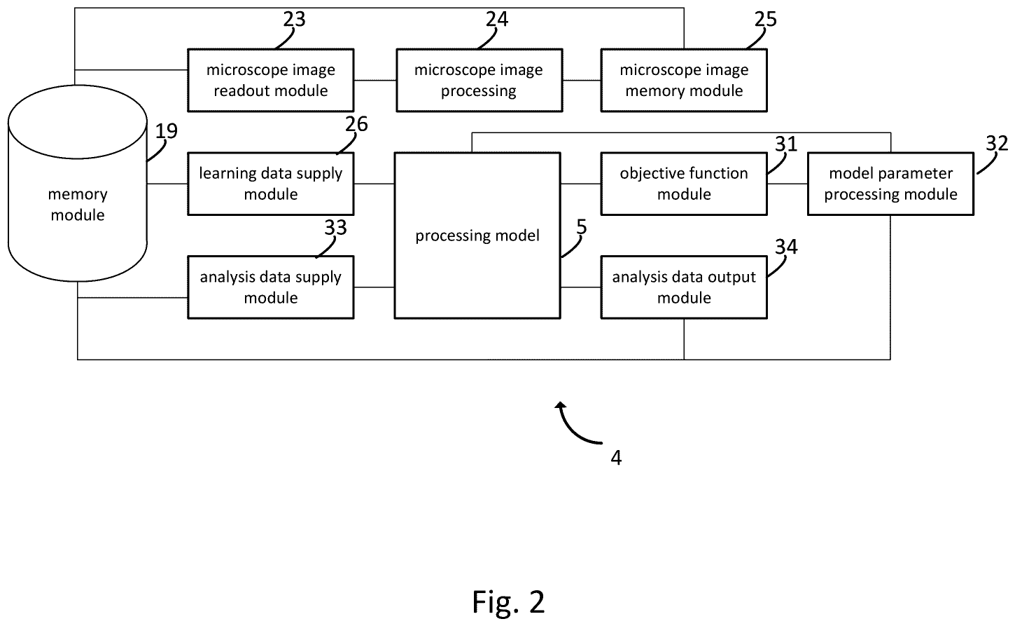

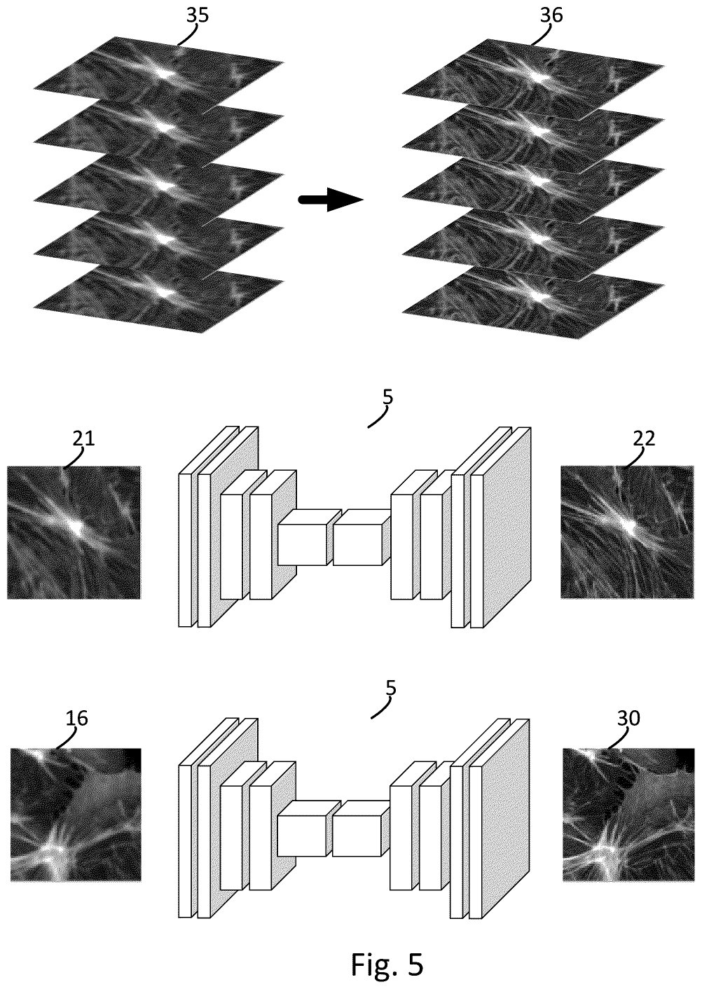

As shown in FIG. 2, the evaluation device 4 also stores training data in the memory module 19, the training data used for training of the processing model 5. The training data comprise an annotated data set 20. The annotated data set 20 contains a learning pair of microscope images consisting of a learning microscope image 21 and a target microscope image 22 (FIG. 5). In the present exemplary embodiment, the learning microscope image 21 is a folded microscope image; a coarse stack according to the first embodiment consists only of the learning microscope image 21.

Methods for optical sectioning with which a suitable microscope can generate clear images of focal planes deep in the interior of a thick sample are known from the prior art. Optical sectioning is used in order to reduce the need for thin sectionings with instruments such as a microtome. In addition, the respective samples are destroyed when thin sectionings are applied, for which reason, for example, a temporal behavior in samples cannot be illustrated. There are a multiplicity of different techniques for optical sectionings, microscopy techniques being used above all in order to improve the quality of optical sectionings.

Good optical sectionings are very popular in microscopy particularly for the reason that they enable a three-dimensional reconstruction of a sample from microscope images which were recorded in different focal planes.

In an ideal microscope, only the light from the focal plane would reach the microscope camera and generate a clear image of the focal plane of the sample. In real microscopes, light from sources outside the focal plane also reaches the microscope camera 12; precisely in the case of thick samples, a considerable amount of material which causes undesired signals can be located between the focal plane and the objective 8 or between the illumination device 13, the focal plane and the objective 8.

In this case, the undesired signals caused depend directly on the structure of the sample under consideration or on the structure of the sample type under consideration. Since the number and diversity of samples under consideration rises increasingly, it is often not possible to provide pre-trained processing models 5 which reliably suppress the undesired signals caused by the samples themselves.

Therefore, with the first embodiment of the present invention provides a machine learning system 1 which, for each new sample type, trains a new processing model 5 on the basis of an independently generated annotated data set 20 for carrying out the deconvolution mapping. In addition to the processing model 5 and the memory module 19, the evaluation device 4 comprises further modules which exchange data via channels 23. The channels 23 are logical data connections between the individual modules. The modules can be designed both as software modules and as hardware modules.

The evaluation device 4 furthermore comprises a microscope image readout module 23. The microscope image readout module 23 reads microscope images 16 from the memory module 19 or from the microscope camera 12 and forwards the microscope images 16 to the microscope image processing module 24. The microscope image processing module 24 processes the microscope images 16 obtained and outputs the processed microscope images 16 to the microscope image memory module 25. The microscope image memory module 25 stores the processed microscope images 16 in the memory module 19.

The evaluation device 4 also comprises a learning data supply module 26 which reads the annotated data set 22 from the memory module 19 and inputs it into the processing model 5.

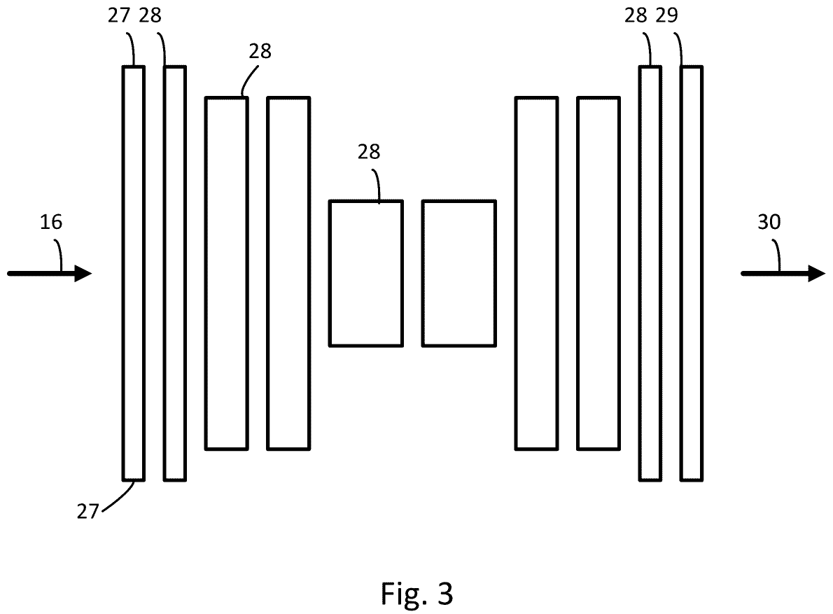

The processing model 5 is a U-Net, that is to say an encoder-decoder network, with an input layer 27, a plurality of intermediate layers 28 and an output layer 29, see FIG. 3. The processing model 5 outputs the resulting microscope images 30 generated from the learning microscope images 21 and the target microscope images 22 to the objective function module 31.

The objective function module 31 receives the resulting microscope images 30 and the target microscope images 22 and calculates an objective function from a resulting microscope image 30 and a corresponding target microscope image 22. The objective function module 31 forwards the calculated objective function to the model parameter processing module 32.

The model parameter processing module 32 receives the objective function from the objective function module 31 and calculates new model parameters for the processing model 5 on the basis of the objective function. The new model parameters are fed back by the model parameter processing module 32 to the processing model 5.

The processing model 5 receives the new model parameters and adapts the model parameters of the processing model 5 on the basis of the new model parameters.

Furthermore, the evaluation device 4 comprises an analysis data supply module 33 which reads microscope images 16 from the memory module 19 for the analysis and forwards them to the fully trained processing model 5. The processing model 5 carries out the fully trained virtual processing mapping using the received microscope images 16. According to the first embodiment, the classic processing mapping corresponding to the virtual processing mapping is an accelerated Richardson-Lucy deconvolution. The processing model 5 forwards the resulting microscope images 30 to the analysis data output module 34. The analysis data output module 34 stores the output resulting microscope images 30 in the memory module 19.

Alternatively, the machine learning system 1 can also consist only of the evaluation device 4. The annotated data set 20 can be transmitted via a communication link or from a mobile data carrier to the memory module 19 of the evaluation device 4, and the evaluation device 4 is then trained on the basis of the training data. If the processing model 5 of the evaluation device 4 is completely trained, the evaluation device 4 can also evaluate microscope images 16 of the sample type independently of a microscope 2 according to the learned processing mapping.

Alternatively to the deconvolution described above, the processing model 5 can also be trained to carry out other virtual processing mappings. For example, the processing model 5 can also carry out an alternative deconvolution mapping. There are many different deconvolution methods which can be used here. These methods can be grouped as follows:
Deblurring methods such as, for example, no neighbor deblurring or unsharp masking
Nearest neighbor method (Castleman, K. R. 1979. Digital image processing. Prentice-Hall, Englewood Cliffs, NJ.)
Accelerated iterative methods, for example Meinel & Edward S. (Origins of linear and nonlinear recursive restoration algorithms, J. Opt. Soc. Am. A 3, 787-799, 1986), Gold's Method (Gold, R. 1964. An Iterative Unfolding Method for Response Matrices. Report no. ANL-6984. Argonne National Laboratory, Chicago.), Jansson-van-Cittert Method (van Cittert, P. H.: “Zum Einfluss der Spaltbreite auf die Intensitätsverteilung in Spektrallinien. II,” Z. Phys. 69, 298-308 (1931); Jansson, P. A. ed.: Deconvolution of Images and Spectra (2Nd Ed.) (Academic Press, Inc., Orlando, FL, USA (1996)).
Richardson-Lucy, iterative method: Richardson-Lucy, classic (Lucy, L. B.: An iterative technique for the rectification of observed distributions, Astron. J., 1974, 79:745-754; Richardson W. H., Bayesian-based iterative method for image restoration. J. Opt. Soc. Am., 1972, 62 (6): 55-59; Richardson-Lucy, accelerated (David S. C. Biggs and Mark Andrews, “Acceleration of iterative image restoration algorithms,” Appl. Opt. 36, 1766-1775, 1997)
Constrained Iterative Maximum Likelihood Method: Schaefer L H, Schuster D, Herz H.: Generalized approach for accelerated maximum likelihood-based image restoration applied to three-dimensional fluorescence microscopy. J Microsc. 2001 November; 204 (Pt 2): 99-107.doi: 10.1046/j.1365-2818.2001.00949.x. PMID: 11737543
Other iterative methods: Agard-Sedat Deconvolution (A. Agard, Yasushi Hiraoka, Peter Shaw, John W. Sedat, Chapter 13 Fluorescence Microscopy in Three Dimensions, Methods in Cell Biology, Academic Press, Volume 30, 1989, Pages 353-377); Blind Deconvolution (Timothy J. Holmes, “Blind deconvolution of quantum-limited incoherent imagery: maximum-likelihood approach,” J. Opt. Soc. Am. J. Micros., 178, 165-181, 1977).

According to one configuration, the microscope 2 can also be a light sheet microscope.

The methods and references listed here are to be understood only by way of example, since there are still further methods which would be equally suitable for the method.

According to the first embodiment, the evaluation device 4 is a computer. Alternatively, however, the evaluation device 4 can also be integrated into the microscope 2 or be realized by means of, for example, a cloud server which makes the evaluation available to a user via a network connection.

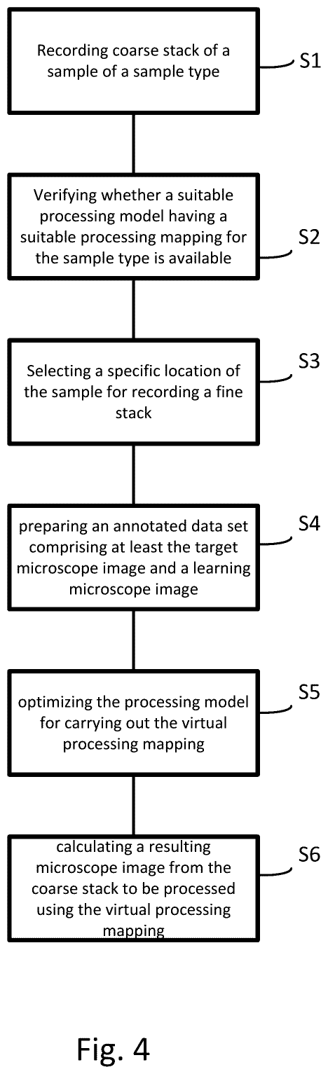

A method for generating a resulting microscope image with a machine learning system 1 having a processing model 5 for microscope images 16 of a sample type according to the first embodiment is explained below (FIG. 4).

The generation of the resulting microscope image 30 with the machine learning system 1 comprises a plurality of steps. According to a first step S1, the microscope camera 12 of the microscope 2 records coarse stacks at a plurality of locations in the sample under consideration, wherein each of the coarse stacks comprises only one microscope image 16. The recorded microscope images 16 are stored in the memory module 19.

According to a second step S2, the evaluation device 4 verifies whether the memory module 19 can provide a suitable processing model having a fully trained processing mapping suitable for the sample type of the examined sample, i.e. in this case a deconvolution mapping.

According to the first embodiment, the evaluation device 4 determines that no suitable processing model 5 is stored in the memory module 19 and instructs the microscope 2 to record a fine stack 35.

In the third step S3, a selection machine learning model independently selects a specific location of the sample on the basis of the sample type. The selection machine learning model has been trained on the basis of specific locations for the recording of fine stacks 35 selected earlier by a user on comparable samples to select the specific location for the recording of fine stacks 35. At the specific location, the microscope 3 automatically records one or more fine stacks 35, which each comprise five microscope images 16 of the sample registered with respect to one another and offset in height. The recorded fine stack 35 is read out from the microscope camera 12 by the microscope image readout module 23.

According to an alternative, a specific location can also be selected by a user, for example, in an overview image.

According to step S4, the microscope image readout module 23 transfers the fine stack 35 to the microscope image processing module 24. The microscope image processing module 24 calculates the so-called learning microscope image 21 for the middle image of the learning stack, utilizing the learning stack, and the target microscope image 22 by means of the deconvolution mapping. Furthermore, the microscope image processing module 24 calculates an annotated data set 20 from the learning pair consisting of the learning microscope image 21 and the target microscope image 22 by means of a wide variety of augmentations, for example, transformations, rotations, stretchings, compressions, grayscale shifts and similar image-to-image transformations, and stores the annotated data set 20 in the memory module 19.

The annotated data set 10 comprises the learning microscope image 21; here, a microscope image of the coarse stack can be used or a plurality thereof, and the target microscope image 22.

In step S5, the learning data supply module 26, the processing model 5, the objective function module 31 and the model parameter processing module 32 carry out a stochastic gradient method for optimizing the model parameters of the processing model 5. For this purpose, the learning data supply module 26 randomly selects a set of examples from the annotated data set 20 in a plurality of successive training steps, inputs the learning microscope images 21 of the randomly selected set of examples into the processing model 5, and the target microscope images 22 to the objective function module. The processing model 5 calculates the resulting microscope images 30 and outputs the resulting microscope images 30 to the objective function module 31. The objective function module 31 determines the objective function for each learning pair, sums it over the learning pairs of the randomly selected set of examples, also referred to as batch, and the model parameter processing module 32 optimizes the processing model 5 on the basis of a gradient determined from the objective function in a gradient descent algorithm.

The optimization of the processing model is carried out with various successive optimization steps until the objective function reaches a termination condition. If the termination condition is reached, the step S5 of training the processing model 5 is ended.

According to step S6, the analysis data supply module 33 reads the coarse stacks recorded according to step S1 from the memory module 19 and inputs the coarse stacks into the fully trained processing model 5. The coarse stacks each comprise only one microscope image 16 of the sample. From the input microscope image 16, the processing model 5 calculates deconvolved microscope images, i.e. the resulting microscope images 30, and outputs the deconvolved microscope images to the analysis data output module 31. The analysis data output module 31 stores the resulting microscope images 30 in the memory module 19.

According to an alternative, step S3 can also be carried out before step S1, step S2 being omitted in this case. Steps S4 and S5 can also be carried out after step S3 and before step S1.

If, for example, it is known that no processing model is yet present, a coarse stack does not first have to be recorded according to step S1, but rather the fine stacks are recorded directly according to step S3, the fine stack is processed by means of the processing mapping according to step S4 and an annotated data set is provided, the processing model is trained according to step S5 and coarse stacks to be processed are subsequently recorded according to step S1.

According to an alternative, the annotated data set 20 stored on the memory module 19 consists only of the learning pair consisting of the learning microscope image 21 and the target microscope image 22. During the training step S5, the learning data supply module 26, for example, then carries out the augmentation described with reference to the first embodiment in each case randomly before input of the learning pair into the processing model 5.

Alternatively, the annotated data set 20 can also comprise more than just one learning pair.

Alternatively, a fine stack 35 can also comprise more than five microscope images 16 offset in height, for example in the case of a sufficient thickness of the sample under consideration. By means of the deconvolution mapping, all microscope images in the fine stack 35, for example, are then deconvolved; a result stack 36 is calculated, wherein the quality of the deconvolution is highest in the middle of the stack (in this respect, see FIG. 5).

If the learning stack comprises a multiplicity of microscope images 16 offset in height and a plurality of the microscope images offset in height can be sufficiently well deconvolved on the basis of the height of the fine stack, the annotated data set can comprise, for example, a fine stack 35 and a target stack, wherein there is a corresponding target microscope image 22 in the target stack for each learning microscope image 21 in the fine stack 35.

According to one configuration of the first embodiment, during the calculation of the target microscope image 22, a plurality of deconvolved candidate microscope images, which are also referred to as decoupled candidate microscope images according to a generalization to other virtual processing mappings, are calculated; the plurality of calculated deconvolved candidate microscope images are compared in order to determine a best of the deconvolved candidate microscope images. The best of the candidate microscope images is selected as the target microscope image. During the calculating of the plurality of deconvolved candidate microscope images a different set of parameters in the deconvolution mapping used is used for each of the plurality of deconvolved candidate microscope images; the used parameters comprise, for example, a used deconvolution algorithm, a number of iterations of the used deconvolution algorithm, used correction methods and correction parameters for the respectively used correction methods.

If a different processing mapping is executed instead of the deconvolution mapping, decoupling mapping can also be referred to in a generalized manner. Herein, a decoupling mapping is understood to mean, for example, a deconvolution mapping, a spectral demixing mapping, a super resolution mapping or also a denoising mapping, also referred to as a descattering mapping.

Correction methods are here correction methods such as the modelling of asymmetrical point spread functions due to spherical aberrations according to the principles of Gibson and Lanni (S. F. Gibson, F. Lanni, “Experimental test of an analytical model of aberration in an oil-immersion objective lens used in three-dimensional light microscopy”, J. Opt. Soc. Am. A, vol. 8, no. 10, pp. 1601-1613 October 1991). Further corrections can address, for example, high background, lamp flickering or bleaching of the dyes.

According to one configuration of the first embodiment, a fully trained processing model 15 is already stored in the memory module 19. According to some embodiments of the present invention, the learned virtual processing mapping is very sensitive to changes in the optical properties of the samples. Therefore, it is not possible for most new samples to use previously trained processing mappings for new samples. However, for example, an experiment with exactly identical samples can run over a plurality of samples, for which reason the plurality of samples have the same sample type.

According to one configuration of the first embodiment, after the recording of all desired microscope images 16 for the analysis of the sample under consideration has been completed in step S1, in step S2, in which it is verified whether a fully trained processing model is stored in the memory module 19, this possibly suitable fully trained processing model is applied to the microscope images 16 recorded in the analysis. The resulting microscope images 30 returned by the analysis data output module 34 to the memory module 19 are thereupon verified with regard to their optical properties, such as, for example, general sharpness impression and geometric state of the deconvolved structures and the absence of artefacts such as structures by incorrect reconstruction. If the resulting microscope images 30 output by the processing model 5 withstand the verification, a renewed recording of a fine stack 35 and the application of the classic deconvolution mapping and a renewed training of a new processing model 5 can be dispensed with and the recorded microscope images 16 can be evaluated directly by means of the found processing model 5, as described above with reference to step S6.

In particular, when verifying the resulting microscope images 30, image artefacts contained or generated in the resulting microscope images 30 are identified and their frequency is verified. If a number or a density with which the image artefacts are generated is too high, a new fine stack 35 then correspondingly has to be recorded and the processing model 5 newly trained. The image artefacts to be identified are in particular: striping, ringing and discontinuous region artefacts.

The image artefacts can be identified either manually or automatically. According to an alternative, a model is trained to distinguish well and poorly reconstructed images from one another.

According to one configuration, initially only one coarse stack to be processed can also be recorded and after the recording of the one coarse stack, it is verified whether a suitably trained processing model is present in a collection of processing models or not.

According to one configuration of the first embodiment, it is conceivable for a previously trained processing model to be provided, for which reason a renewed recording of fine stacks 35 and a renewed training can be avoided.

According to one configuration of the first embodiment, a coarse stack comprises a plurality of microscope images 16 of the fine stack 35. A distance of the microscope images 16 offset in height of the coarse stack is in this case larger than a distance of the microscope images 16 offset in height in the fine stack 35. Every second of the microscope images 16 offset in height of the fine stack 35 is transferred into the coarse stack, with the result that a distance of the microscope images 16 offset in height in the coarse stack is twice as large as in the fine stack 35. Analogously, the coarse stack would also comprise a plurality of microscope images offset in height, with the same number of microscope images 16 offset in height and the same distance as in the fine stack 35.

According to an alternative, only every third, only every fourth, or only every fifth of the microscope images 16 offset in height of the fine stack 35 is transferred into the coarse stack of the annotated data set 20.

If a coarse stack with more than one microscope image 16 is used in the annotated data set 20, then the quality of the virtual deconvolution mapping can thus be used without excessively damaging a sample as a result of further recordings.

According to one configuration of the first embodiment, the microscope 2 can also be a laser scanning microscope with a wide-open pinhole.

A second embodiment differs from the first embodiment in that the fine stack and the coarse stack are each lambda stacks. The lambda stacks can be 2D lambda stacks, but in particular they can also be 3D lambda stacks, i.e. the lambda stacks image the sample in the three spatial dimensions and also correspondingly spectrally resolved.

According to a first configuration of the second embodiment, the microscope 2 is a confocal microscope with a laser scanner as illumination device 13. The laser scanner comprises a tunable laser which can be tuned according to the examined sample and the fluorophores contained in the sample over the spectral range of interest for the fluorescences which occur. For example, the laser can be tuned over the spectral range of interest such that the laser can be set to 5, 10, 20, 30 or even 40 different wavelengths within the spectral range of interest and can thus excite fluorescences in the sample with different wavelengths over the spectral range of interest.

The spectral range of interest is understood to mean the range of the spectrum in which fluorescences occur, in particular in which excitation and emission take place. In particular, excitations and emissions should take place in a significant proportion of the excitations and emissions which occur overall. For example, the spectral range of interest captures overall for example 80%, 85%, 90% or else 95% of the excitations and/or emissions which occur overall.

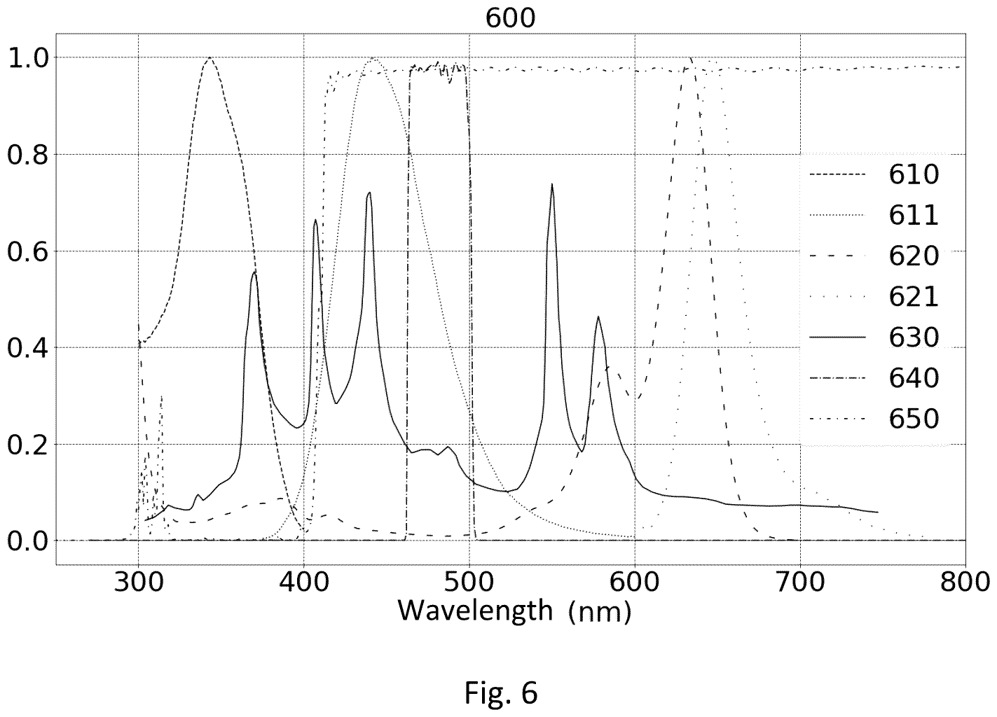

For example, FIG. 6 shows different microscopy spectra as are used in the microscopy of fluorescent samples. A first absorption spectrum 610 and a first emission spectrum 611 of a first fluorophore extend between 300 and 600 nm in this example. A second absorption spectrum 620 and a second emission spectrum 621 of a second fluorophore extend between approximately 400 and 700 nm. A first continuous spectrum 630 of a halogen metal vapor lamp is recorded here between 300 and 700 nm. A first transmission spectrum 640 of a filter is transmissive for wavelengths in the range between approximately 460 and 500 nm. A second transmission spectrum 650 of a dichroic mirror reflects wavelengths up to approximately 410. The spectral range of interest extends for this example over the region in which excitation and deexcitation of the fluorophores occur, that is to say for this example approximately between 300 and 700 nm. The spectra drawn in are in each case to be understood only by way of example. The number of different available fluorophores or fluorophores suitable for the respective experiment or the respective sample, and their respective characteristic spectra, are known to the person skilled in the art and can be looked up in different databases known to the person skilled in the art. The same applies to the commonly available filters, dichroic mirrors, light sources and detectors.

Moreover, the imaging device according to the second embodiment comprises a multi-channel photomultiplier instead of the microscope camera 12. A pinhole aperture and a diffraction grating are arranged between the sample and the multi-channel photomultiplier. The diffraction grating spectrally decomposes the light emitted by the sample. A photocathode of the multi-channel photomultiplier is divided into segments in accordance with the number of channels of the multi-channel photomultiplier. Since the imaging device according to the second embodiment has the diffraction grating in the beam path upstream of the multi-channel photomultiplier, which spectrally decomposes fluorescence light emitted by the sample, each of the segments of the photocathode captures a (different) partial region of the spectrum of the fluorescence spectrum of the sample.

Step S3, in which the specific location of the sample is selected and the fine stack is recorded, according to the second embodiment differs from the first embodiment in that a lambda stack, also called A stack, is recorded instead of a z-stack with microscope images 16 offset in height. A lambda stack according to the second embodiment comprises a multiplicity of microscope images 16 registered to one another, wherein a different partial region of the spectrum, also called spectral range, of the fluorescence image is recorded in each of the microscope images 16.

According to the second embodiment, the laser scanner is moved point by point over the sample during the recording of the lambda stack. At each point of the sample, the laser of the laser scanner is controlled such that the laser illuminates the sample with different wavelengths, for example 5, 10, 20, 30 or 40 different wavelengths, such that the fluorophores contained in the sample are excited with different wavelengths. For each point of the sample, a fluorescence spectrum with a number of spectral ranges corresponding to the number of channels of the multi-channel photomultiplier (for example 4, 8, 16 or 32 or any other desired number of channels) is recorded with the multi-channel photomultiplier. Each of the segments of the photocathode or each channel of the multi-channel photomultiplier thus supplies an image signal, also called a fluorescence signal, corresponding to the point in the sample and the respective spectral range. The image signals via the points of the sample of the respective spectral range thus each form a microscope image 16 of the lambda stack for the respective spectral range according to the second embodiment.

Step S4 of the second embodiment differs from step S4 of the first embodiment in that the fine stack 35 is a lambda stack and the processing mapping or the decoupling mapping is a spectral demixing mapping which comprises linear demixing of the lambda stack. During linear demixing, a relative proportion of the respective fluorophore in the spectrum of the respective pixel or of the respective microscope image is determined for each fluorophore for each pixel of the microscope image 16. In this case, autofluorescent objects occurring in the sample are treated as a further fluorophore and can be deducted as background objects after the linear demixing for each pixel of the microscope image 16.

According to the second embodiment, a target microscope image 22 is determined by means of the demixing mapping, wherein the proportions of the fluorophores contained in the sample in the respective image signal are respectively assigned to the image signals of each image point of the target microscope image. Correspondingly, it can then be selected for the target microscope image, for example, that only proportions of specific ones of the fluorophores contained in the sample are respectively displayed on the screen 18 of the control apparatus 3. For example, the control apparatus 3 can be configured not to automatically display a signal proportion of the autofluorescent objects contained in the sample.

Alternatively, the target microscope image can also comprise a plurality of images, wherein each of the images respectively comprises only the proportion of one of the fluorophores contained in the sample and in particular also one of the images comprises the proportion of the autofluorescence in the sample.

According to the second embodiment, the coarse stack comprises a part of the microscope images 16 of the lambda stack, for example every third of the microscope images 16 of the lambda stack. Correspondingly, a step S4 of preparing the annotated data set comprises selecting the microscope images 16 from the fine stack for the coarse stack.

According to one configuration of the step S4 of preparing the annotated data set, the coarse stack is prepared by recording a further lambda stack with a coarser spectral granularity. A lambda stack then has a coarser spectral granularity if, for example, the illumination device travels over an excitation spectrum with a small granularity. If, for example, during the recording of the fine stack 35, the laser is tuned such that the sample is excited to fluoresce with a total of 40 different wavelengths, then, for example, during the recording of the coarse stack, the sample can be excited to fluoresce only with every second wavelength of the wavelengths used during the recording of the fine stack 35, for which reason a granularity and thus a spectral granularity is coarser.

According to one configuration of the second embodiment, during the recording of the coarse stack, a coarser spectral granularity can be achieved by interconnecting segments of the anode of the multi-channel photomultiplier such that the multi-channel photomultiplier has, for example, only one half, one third or only one quarter of the original channels, in which in each case 2, 3 or 4 neighboring channels of the multi-channel photomultiplier are read out jointly.

According to a second configuration of the second embodiment, instead of the laser scanner, an illumination device 13 consisting of one or more light sources can be used during the recording of the lambda stacks, that is to say of the coarse stack and of the fine stack. According to the second configuration, the microscope 2 can be the confocal microscope of the first configuration of the second embodiment, but the microscope 2 of the first embodiment can also be used or a reflected light microscope. The light sources can be, for example, a plurality of LEDs which each emit monochromatic light; alternatively, for example, only one broadband light source can be used; furthermore, alternatively, a plurality of broadband light sources can also be used as the exposure device 13.

If a plurality of LEDs are used as the exposure device 13, then, during the recording of the fine stacks, for example, for each of the microscope images 16 of the fine stack, in each case one LED can be used for exposing the sample, a resulting fluorescence spectrum is then recorded, for example, using a microscope camera 12, this procedure is repeated for each of the LEDs, and each of the recorded microscope images 16 forms one of the microscope images 16 of the fine stack 35. The coarse stack can then be created, for example, by selecting a subset of the microscope images 16 of the fine stack.

If, for example, a broadband light source is used as the exposure device 13, then, in addition, filters in the beam path can either filter the excitation spectrum of the light source such that always only a partial region of the broadband spectrum of the light source impinges on the sample; for the capturing of the fine stack, a plurality of filters are correspondingly used which each filter out other partial regions of the broadband spectrum; for each of the plurality of filters, in each case one microscope 16 of the fine stack 35 is recorded.

As a further variation of the second configuration, instead of the excitation spectrum of the light source, the fluorescence spectrum of the sample can also be filtered by means of different filters such that different filters each filter out different partial regions of the fluorescence spectrum again and in each case one microscope image 16 of the fine stack 35 is recorded for each of the different partial regions.

As already described above with reference to the first configuration of the second embodiment, the coarse stack can be determined by selection of microscope images 16 from the fine stack 35. According to an alternative, however, the coarse stack can also be prepared by means of further recordings of the sample. For example, during the capturing of the coarse stack, other broadband filters can be used for filtering either the excitation spectrum or the fluorescence spectrum of the sample. If broadband filters are used during the capturing of the coarse stack, then, for example, recording times can be reduced.

According to the second embodiment, step S1 of recording the coarse stack differs from the first embodiment in that a lambda stack is recorded and no z-stack with microscope images 16 offset in height. In contrast to the recording of the fine stack 35, which is used for the classic spectral demixing mapping, only partial regions of the spectrum are scanned with the laser of the laser scanner during the recording of the coarse stack in step S1.

If, for example, during the recording of the fine stack, the tunable laser is operated such that the sample is excited with for example 20 different wavelengths over the spectral range of interest, then the laser of the laser scanner is actuated during the recording of the coarse stack such that the sample is excited in each case only with ten different wavelengths over the spectral range of interest. The sample is therefore excited only with in each case every second wavelength of the wavelengths used during the recording of the fine stack. Since some of the wavelengths previously used during the recording of the fine stack for exciting the sample are no longer used, the sample can be preserved during the inference; it is thus no longer loaded to such an extent.

For example, only every third, every fourth or every fifth wavelength of the wavelengths can also be used for excitation. Likewise, the laser can also be tuned with different, non-integer step sizes.

In step S5, the processing model 5 is trained with the annotated data set 20 consisting of the coarse stack and the target microscope image 22. According to the second embodiment, the learning data, i.e. the coarse stack and the target microscope image, are likewise augmented again in order to provide a large number of learning data for training the processing model 5.

According to one configuration of the second embodiment, the coarse stack comprises every second, every third, every fourth, every fifth or any other desired one of the microscope images 16 of the fine stack 35. According to another configuration, certain particularly significant partial regions of the spectrum which are as suitable as possible for linear demixing are selected from the lambda stack and captured in the coarse stack.

According to one configuration of the second embodiment, instead of the laser scanner, an illumination device with a discrete or continuous, but known spectrum can also be used. If an illumination device with a continuous spectrum is used, during the recording of the coarse stack, the spectrum is filtered with suitable filters in order to filter out the spectral component required for the excitation of the dyes. Such illumination devices can be, for example, light-emitting diode (LED)-based or else metal arc lamps.

According to one configuration of the second embodiment, during the recording of the coarse stack, a plurality of neighboring segments of the photocathode of the multi-channel photomultiplier can each be combined. For example, in each case two, three, four, five or more. As a result, the number of spectral ranges in the coarse stack is reduced and a recorded data volume or a data volume to be processed can thus be reduced.

According to the second embodiment, the processing model is again an aggregate processing model and the aggregate processing model is trained to carry out the virtual processing mapping in one step. Alternatively, however, as described with reference to the first embodiment, the processing model can be a stage processing model in which a decoupling model learns the classical processing mapping, here the spectral demixing mapping, while the detail enhancement model either learns a mapping from the coarse stack to the fine stack or a mapping from a decoupled coarse stack to the decoupled fine stack or from a coarsely spectrally resolved microscope image to a finely spectrally resolved microscope image, the decoupling mapping is correspondingly applied to the coarse stack or to the fine stack created by means of the detail enhancement model.

According to the third embodiment, in contrast to the first embodiment, the processing mapping is a denoising mapping.

In step S3 according to the third embodiment, the fine stack comprises a plurality of microscope images 16 of the sample, wherein the microscope images 16 are registered to one another and have each been recorded with the same recording parameters such as, for example, focus, zoom, exposure time, illumination intensity, illumination spectrum. The fine stack comprises at least three, or at least five, or at least ten, or at least 15 microscope images 16.

According to step S4 of the third embodiment, a denoising mapping is applied to the microscope images of the fine stack, by means of which a denoised target microscope image 22 is calculated from the fine stack. This can be calculated, for example, by averaging, by adding or similar classical processing mappings.

In step S4, individual ones of the microscope images 16, for example every second, every third, every fourth or every fifth of the fine stack, are selected and transferred into the coarse stack. Alternatively, any selection of the microscope images 16 of the fine stack 35 can also be transferred into the coarse stack, for example one half, one third, one quarter or one fifth.

In step S5 of the third embodiment, by augmenting the coarse stack and the target microscope image 22, the number of the microscope images to be input into the processing model 5 for training the processing model 5 is increased as described above in order to train the processing model 5 for executing the denoising mapping.

In step S1, a coarse stack is recorded, wherein the coarse stack is recorded under the same recording conditions as the microscope images 16 of the fine stack. The microscope images 16 of the fine stack therefore differ from the microscope images 16 of the coarse stack only by a random image noise. The trained virtual processing mapping according to the third embodiment generates a denoised resulting microscope image from the few images of the coarse stack, the signal-to-noise ratio of which corresponds approximately to that of the target microscope image 22. Since only a few recordings of the sample are in turn required for recording the coarse stack, the processing mapping learnt according to the third embodiment can also significantly reduce a sample loading.

The denoising mapping is preferably implemented by means of the aggregate processing model.

However, according to one configuration, the denoising mapping can also be implemented by means of a stage processing model. In particular, a detail enhancement model can execute a detail enhancement mapping which determines a fine stack from a coarse stack. The denoising mapping is applied to the resulting fine stack as the decoupling mapping.

Alternatively, the decoupling mapping can also be applied first and the detail enhancement model subsequently determines the finely denoised microscope image, also referred to as fine decoupling stack, on the basis of the coarsely denoised microscope image, also referred to as decoupling coarse stack, determined from the coarse stack by means of the decoupling mapping. The finely denoised microscope image is then again the resulting microscope image as described above in step S5. A plurality of finely denoised microscope images can also always be calculated.

According to this embodiment, a method is provided in which the resulting microscope images determined on the basis of a recorded coarse stack have approximately the same quality, i.e. approximately the same signal-to-noise ratio, as the target microscope image determined on the basis of the fine stack.

A fourth embodiment differs from the preceding embodiments in that the microscope images of the fine stack and coarse stack are each recorded using a structured illumination pattern, and the image stacks recorded using the structured illumination pattern are reconstructed by means of a super resolution mapping to form a super resolved microscope image 704. That is to say, according to the fourth embodiment, the virtual processing mapping comprises a super resolution mapping.

The fourth embodiment differs from the preceding embodiments in that the exposure device is designed to generate the structured illumination pattern. Exposure devices which generate structured illumination patterns are well known from the prior art; they use, for example, lasers whose light is diffracted by means of diffraction gratings to form a structured illumination pattern.

A structured illumination pattern can be a stripe pattern, a point pattern, a grating pattern, a line grid, a square point grid or a hexagonal point grid. A stack in each case comprises microscope images in which an orientation and/or a position of the illumination pattern in or on the sample has been selectively varied between different ones of the microscope images. In this case, the stacks can be both 2D and 3D recordings of the sample; for 2D recordings, the position and/or orientation of the structured illumination pattern in the focal plane is varied, while for 3D patterns the position and/or orientation of an entire volume of a sample to be recorded is varied.

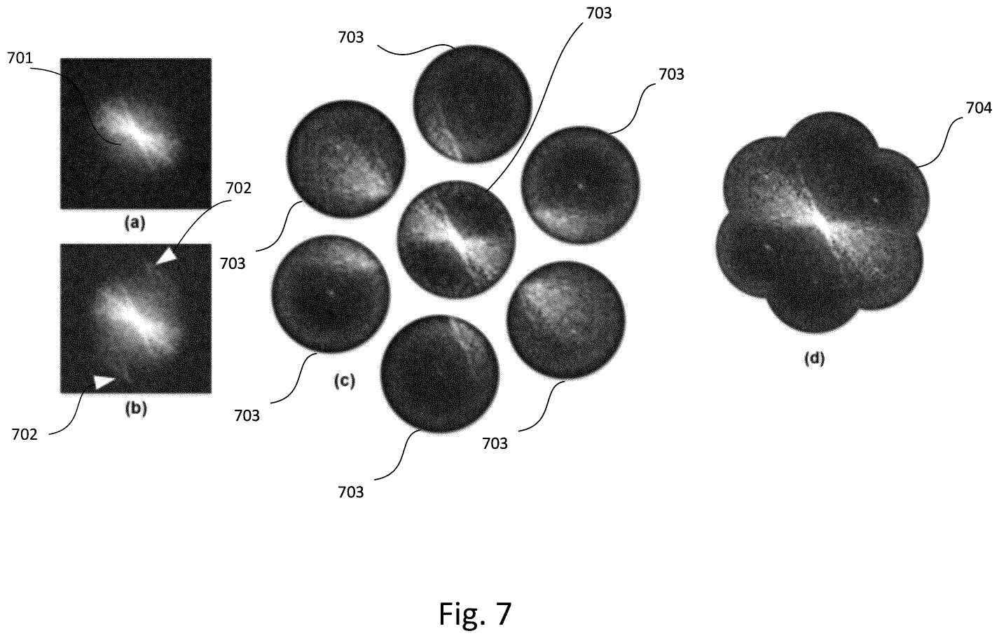

An imaging device has an optical resolution limit due to diffraction which occurs, wherein structures whose distance in a sample is smaller than the resolution limit can no longer be reproduced separately from one another by the imaging device. If this phenomenon is transferred into the Fourier space, this is also referred to as a cutoff frequency; an imaging device does not transmit frequency components above the cutoff frequency, which is why there is blurring of actually sharp structures due to the optical resolution limit or the cutoff frequency, see, for example, the blurred structure 701 in FIG. 7 (a).

When using structured illumination patterns, overlaps of the structured illumination pattern with structures of the sample are formed; so-called Moiré patterns 702 are formed in the microscope images. Moiré patterns 702 are always formed when structures of (slightly) different frequencies and/or orientation overlap. The Moiré patterns 702 are relatively large structures, i.e. structures larger than the resolution limit of the imaging device, in which superresolution information of the sample is encoded, i.e. information about structures smaller than the resolution limit corresponds to the frequencies large in the Fourier space above the cutoff frequency. Such Moirè patterns 702 are marked with the white arrows in FIG. 7 (b), for example. The superresolution information contained in a single microscope image is not sufficient to reconstruct the recorded (high-frequency) structures therefrom; for this purpose, a plurality of microscope images each with a different phase position or orientation of the structured illumination pattern with respect to the sample have to be recorded.

According to the present example, microscope images were recorded for in each case three different orientations and phase positions. Depending on the exposure parameters used, the nine images form the fine stack or the coarse stack. Seven high-frequency components 703 can then be reconstructed from the new recordings; these are illustrated by way of example in FIG. 7 (c). The coarse and fine stacks of the fourth embodiment now each comprise microscope images which have a different phase position with respect to the sample by varying, for example shifting or rotating, the structured illumination pattern, for which reason they are also called phase images. On the basis of the known illumination pattern, the components can be calculated from the phase images and a superresolved microscope image 704 can be calculated from the components, as illustrated in FIG. 7 (d).

It should be noted that the structure illustrated in the reconstructed superresolved microscope image 704 in FIG. 7 (d) is approximately twice as large as the blurred structure in the microscope image in FIG. 7 (a), but the two structures are identical, i.e. the resolution has approximately doubled as a result of the superresolution mapping.

Different methods for evaluating and calculating superresolved microscope images from a stack of phase images are known in the prior art. Two of the methods, SIM and dual iterative SIM, diSIM, are described, for example, in “Super-Resolution Imaging by Dual Iterative Structured Illumination Microscopy” by Anna Löschberger, Yauheni Novikau, Ralf Netz, Marie-Christine Spindler, Ricardo Benavente, Teresa Klein, Markus Sauer, Dr. Ingo Kleppe (bioRxiv 2021.05.12.443720; doi: https://doi.org/10.1101/2021.05.12.443720).

Step S1 of the fourth embodiment differs from step S1 of the first embodiment in that phase images with different phase positions of the structured illumination pattern on the sample are recorded during the recording of the coarse stack, wherein the phase images have a greater noise with respect to the fine stack, i.e. during the recording of the coarse stack, the recording parameters are selected such that either an illuminance of an illumination device 13 is selected to be lower than during the recording of the fine stack, or an illumination time is shortened with an unchanged illuminance, such that the phase images of the coarse stack have a lower signal-to-noise ratio with respect to the phase images of the fine stack.

Step S3, in which the specific location of the sample is selected and the fine stack is recorded, according to the 4th embodiment differs from the 1st embodiment in that instead of a Z-stack a stack with phase images is recorded. When recording the fine stack, recording parameters of the imaging device 20 are selected such that the phase images have only a low noise, in particular a higher signal-to-noise ratio than the phase images of the coarse stack.

Step S4 of the fourth embodiment differs from step S4 of the first embodiment in that the fine stack 35 comprises phase images and the processing mapping is a super resolution mapping which calculates a super resolved microscope image from the plurality of phase images of the fine stack 35. The super resolved microscope image is used as target microscope image in the following step S5.

Step S5 of the fourth embodiment differs from step S5 of the first embodiment in that the processing model is trained for carrying out the super resolution mapping. For the training, the coarse stack is input into the processing model; the output of the processing model, the resulting image, is compared with the target microscope image, in this case the super resolved microscope image, in order to calculate the objective function.

In principle, it would be possible to calculate the super resolved microscope image from the coarse stacks. On account of the lower signal-to-noise ratio of the phase images of the coarse stack compared with the phase images of the fine stack and because the superresolution information is weaker compared with the information within the resolution limit, the quality of the reconstructions of the super resolved microscope image from the phase images of the coarse stack is reduced quite considerably compared with the reconstruction from the fine stack. Therefore, the inventors propose to train a processing model depending on the sample type to reconstruct a super resolved microscope image with a high quality also from a coarse stack. For this purpose, an annotated data set is automatically generated in each case for each sample type; thus, the processing model can be individually taught for each sample type to reconstruct corresponding structures occurring individually in the respective sample type with a good quality.

The fourth embodiment differs from step S6 of the first embodiment in that the processing model receives the coarse stacks, in this case the coarse stacks with phase images, as input and calculates super resolved microscope images from the coarse stacks.

According to configurations of the fourth embodiment, the coarse stack can be a 2D stack or also a 3D stack. If the coarse stack of the fourth embodiment is a 3D stack, the processing model can output in particular a super resolved stack; in particular, a deconvolution mapping can also be applied to the super resolved stack after the output of the super resolved stack. Alternatively, the processing model according to the fourth embodiment can be directly trained to deconvolve the super resolved stack, i.e. the processing model directly outputs a deconvolved, super resolved stack.

A fifth embodiment differs from the first four embodiments in steps S4 to S6 in that the processing model is not an aggregate processing model, as in the embodiments described above, but rather a stage processing model. In a stage processing model, the virtual processing mapping is not executed by an aggregate processing model, but rather by two different processing models. A decoupling model executes the classical processing mapping, in this case therefore the deconvolution mapping, the super resolution mapping, the spectral demixing mapping or the denoising mapping, the detail enhancement model executes a corresponding detail enhancement, for example from the coarse stack to the fine stack, a fine stack generated by means of the detail enhancement model is then classically processed using the decoupling model, i.e. the decoupling model does not have to be trained in a supervised manner, for instance, but rather executes the classical processing mapping directly. Alternatively, the detail enhancement model can also be trained such that it maps a decoupling coarse stack determined from the coarse stack by means of the decoupling mapping to the target microscope image, wherein the target microscope image was determined from the fine stack by means of the decoupling mapping.

The stage processing model can be applied to all the embodiments described above with its different processing mappings. The same also applies correspondingly in the inference; a stage processing model can also be applied in the inference.

According to a sixth embodiment, the microscope is a light sheet microscope. In the case of a light sheet microscope, only one thin sheet is illuminated in the sample by means of a special illumination device. The thin sheet is also referred to as a light sheet or light disk.

In contrast to the arrangements described above, in the case of a light sheet microscope, an objective arrangement and a microscope camera 12 are substantially perpendicular to a plane lying in the light sheet, i.e. an illumination direction of the light sheet and an observation direction are perpendicular to one another.

Steps S1 and S3 according to the sixth embodiment differ from the preceding embodiments in particular in that, during the recording of an image stack offset in height with a light sheet microscope, a so-called light sheet stack, in contrast to the recording of an image stack offset in height, for example with a wide-field microscope, not only the focal position of the objective in the sample is displaced, but, in addition to the focal position of the objective of the light sheet microscope, the position of the light sheet in the sample is also displaced, wherein the focal position and the position of the light sheet in the sample are compared or displaced jointly in the sample such that the focal position of the objective is always illuminated by the light sheet.

According to the embodiments described above, the microscope can be not only a wide-field microscope; alternatively, a laser scanning microscope, LSM, a light sheet microscope, also referred to as light disk microscope, a spinning disc microscope, phase contrast microscope, bright-field or dark-field microscope, any form of fluorescence microscope or a STED microscope can also be used.

The configurations of the embodiments described in each case with reference to the embodiments described above can also be applied in each case analogously to the other embodiments in each case.

### LIST OF REFERENCE SIGNS

1 Machine learning system
2 microscope
3 control apparatus
4 evaluation device
processing model
6 stand
7 objective turret
8 mounted objective
9 sample stage
holding frame
11 sample carrier
12 microscope camera
13 illumination device
14 overview camera
field of view
16 microscope image
17 mirror
18 screen
19 memory module
annotated data set
21 learning microscope image
22 target microscope image
23 microscope image readout module
24 microscope image processing module
microscope image memory module
26 learning data supply module
27 input layer
28 intermediate layers
29 output layer
resulting microscope images
31 objective function module
32 model parameter processing module
33 analysis data supply module
34 analysis data output module
35 fine stack
36 result stack
600 microscopy spectra
610 first absorption spectrum
611 first emission spectrum
620 second absorption spectrum
621 second emission spectrum
630 first continuous spectrum
640 first transmission spectrum
650 second transmission spectrum
701 blurred structure
702 Moiré pattern
703 high-frequency component
704 super resolved microscope image

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for training a machine learning system having a processing model for a sample type, which processes microscope images of samples of the sample type by virtual processing mapping, comprising:
recording at least one fine stack of a sample of the sample type, wherein the at least one fine stack comprises microscope images of the sample registered with respect to one another,
determining at least one target microscope image based on the fine stack and the virtual processing mapping,
preparing an annotated data set comprising at least the target microscope image and a learning microscope image, wherein the learning microscope image is based on a coarse stack capturing the sample coarser than the fine stack and in particular has more image artefacts than the target microscope image,
optimizing the processing model for carrying out the virtual processing mapping on the basis of the annotated data set of the sample type.

2. The method according to claim 1, further comprising recording the coarse stack.

3. The method according to claim 1, wherein the recording of the at least one fine stack of microscope images is carried out at a specific location of the sample, in particular the specific location is not needed for recording further microscope images of the sample, and in particular the specific location of the sample is automatically selected by the machine learning system, for example in a predetermined region of the sample.

4. The method according to claim 3, wherein prior to recording the fine stack, the method comprises identifying one or more objects to be examined in the sample and controlling the machine learning system to record the fine stack based on the identified objects to be examined in the sample.

5. The method according to claim 1, wherein the training of the processing model is either a learning from scratch of the processing model or a transfer learning of a pre-trained processing model, in particular the pre-trained processing model is selected from a number of pre-trained processing models on the basis of the sample type, in particular the pre-trained processing models have been pre-trained on the basis of an in-domain data set or have been pre-trained on the basis of an out-of-domain data set.

6. The method according to claim 1, wherein the processing model comprises a neural network, in particular a fully convolutional network or a patch-based network, an encoder-decoder network, in particular a U-Net, a generator of a generative adversary network or a transformer.

7. The method according to claim 1, wherein the processing model is a stage processing model or an aggregate processing model, wherein the stage processing model comprises a detail enhancement model and a decoupling model, wherein the detail enhancement model is trained by the annotated data set to execute a detail enhancement mapping and the decoupling model classically calculates a decoupling mapping and the aggregate processing model is trained by the annotated data set to execute the detail enhancement mapping and the decoupling mapping in one step, the annotated data set to train the stage processing model comprises either the microscope images of the coarse stack as the learning microscope image and the microscope images of the fine stack as the target microscope image or comprises a decoupling coarse stack comprising at least one microscope image determined from the coarse stack by the decoupling mapping as the learning microscope image and comprises a decoupling fine stack comprising at least one microscope image, the decoupling fine stack determined from the fine stack by the decoupling mapping as the target microscope image, and the annotated data set for training the aggregate processing model comprises at least one microscope image of the coarse stack as the learning microscope image and at least one decoupled microscope image determined from the fine stack by the decoupling mapping as the target microscope image.

8. The method according to the preceding claim 7, wherein the virtual processing mapping independently of the sample and the sample type in particular is one or more of:
a deconvolution mapping,
a super resolution mapping,
a spectral demixing mapping,
an artifact removal mapping,
a denoising mapping, or
a descattering mapping.

9. The method according to claim 7, wherein the decoupling mapping is a deconvolution mapping, the microscope images of the fine stack and the coarse stack are offset in height with respect to one another, a distance of the microscope images offset in height with respect to one another is smaller in the fine stack than in the coarse stack, or the coarse stack comprises fewer microscope images than the fine stack, in particular the coarse stack is a strict subset of the fine stack, and the coarse stack thus captures the sample coarser than the fine stack.

10. The method according to claim 9, wherein the distance of the microscope images offset in height is selected, for example, depending on the sample type and/or depending on context information, in particular the method comprises determining the distance of the microscope images offset in height in the fine stack and/or in the coarse stack, in particular the distance is determined depending on the sample type and/or depending on the context information such that the deconvolution mapping can be carried out, and the recording of the fine stack is carried out automatically on the basis of the determined distance.

11. The method according to claim 9, wherein the deconvolution mapping uses a depth-variant point spread function.

12. The method according to claim 7, wherein the decoupling mapping is a spectral demixing mapping, the fine stack and the coarse stack are each lambda stacks, wherein the different microscope images of a lambda stack each capture a different spectral range of a spectrum, in particular a continuous spectrum, the microscope images of the coarse stack capture the spectrum coarser than the microscope images of the fine stack, in particular the coarse stack comprises fewer microscope images than the fine stack, and/or in particular the coarse stack captures the captured spectrum coarser than the fine stack.

13. The method according to the preceding claim 12, wherein the capturing of the spectrum is adapted by one or more of:
varying the excitation spectrum for excitation of fluorophores contained in the sample, in particular the excitation spectrum is a continuous spectrum and/or a discrete spectrum, and in particular the excitation spectrum is varied such that the different excitation spectra used capture the spectrum coarser or finer;
varying filters used in the beam path of an image capturing device between the capturing of the fine stack and the coarse stack, which filter the excitation spectrum and/or the fluorescence spectrum, in particular filters with different bandwidths can be used, in particular filters with narrower bandwidths are used during the capturing of the fine stack than during the capturing of the coarse stack, or fewer spectral ranges are captured during the capturing of the coarse stack than during the capturing of the fine stack;
combining a plurality of color channels of the fine stack to form a color channel of the coarse stack; or
combining a plurality of microscope images of the fine stack to form a microscope image of the coarse stack.

14. The method according to claim 7, wherein the decoupling mapping is a denoising mapping, the fine stack comprises a plurality of noisy microscope images recorded with the same recording parameters, and the denoising mapping calculates a denoised target microscope image from the plurality of noisy microscope images in the fine stack and selects a strict subset of the noisy microscope images of the fine stack as a coarse stack or records a coarse stack at the same location in the sample with fewer microscope images.

15. The method according to the preceding claim 7, wherein the decoupling mapping is a super resolution mapping, and the recording of the fine stack and the coarse stack comprises illuminating the sample with a structured illumination pattern and changing the illumination pattern on the sample such that a phase position of the illumination pattern in the sample is different for different microscope images of a stack, in particular an exposure time during the recording of the coarse stack is shorter than during the recording of the fine stack, or illumination intensity during the recording of the coarse stack is lower than during the recording of the fine stack, such that the microscope images of the coarse stack have a lower signal-to-noise ratio than the images of the fine stack, and the coarse stack thus captures the sample coarser than the fine stack.

16. The method according to the preceding claim 15, wherein the structured illumination pattern in particular comprises one or more of a line grid, a point grid, a square point grid or a hexagonal point grid, and the varying of the structured illumination pattern comprises shifting the phase position in the sample and/or changing the orientation of the structured illumination pattern.

17. The method according to claim 15, wherein the illuminating with the structured illumination pattern comprises mixing high-frequency components of the structured illumination pattern with high-frequency components of structures in the sample, different mixed high-frequency components are formed by shifting the phase position of the illumination pattern respectively depending on the phase position of other high-frequency components of structures of the sample with other high-frequency components of the illumination pattern, the different mixed high-frequency components are captured in different ones of the microscope images of a stack, and the calculating of the target microscope image comprises demixing the different mixed high-frequency components by the super resolution mapping in order to calculate the super resolution microscope image.

18. The method according to claim 15, wherein the demixing comprises deconvolution using a point spread function, wherein the point spread function is a filtered point spread function by certain ones of the mixed high-frequency components are filtered out.

19. The method according to claim 7, wherein the determining of the at least one target microscope image from the fine stack with the decoupling mapping comprises calculating one or more decoupled candidate microscope images and selecting the target microscope image from the plurality of decoupled candidate microscope images, wherein in the calculating of the plurality of decoupled candidate microscope images a different set of parameters of the decoupling mapping is used for each of the plurality of decoupled candidate microscope images, wherein by the used parameters for example a respectively used decoupling algorithm, a number of iterations of the used decoupling algorithm, used correction methods and correction parameters of the used correction method are selected.

20. The method according to claim 1, wherein the determining of the at least one target microscope image comprises verifying the at least one target microscope image which determines whether the decoupling of the learning microscope image was successful.

21. The method according to claim 1, wherein the determining of the at least one target microscope image comprises calculating a target stack.

22. The method according to claim 1, wherein the annotated data set comprises a plurality of target microscope images and a corresponding learning microscope image for each of the target microscope images.

23. The method according to claim 1, wherein the optimizing of the processing model comprises augmenting the annotated data set or simulating further data utilizing a point spread function and the target microscope image of the annotated data set, wherein the point spread function is, for example, a depth-variant point spread function.

24. The method according to claim 1, wherein the augmenting in particular comprises, before the calculating of the at least one target microscope image, one or more of:
transforming the microscope images of the fine stack, wherein the transforming of the microscope images of the fine stack comprises one or more of:
denoising,
de-blooming,
mirroring,
rotating,
scaling,
deforming by an elastic grid,
brightening,
darkening,
adjusting the gamma correction value,
vignetting,
an offset,
color inversion,
artificial noise,
sub-sampling,
masking,
blurring,
any filtering with a linear or non-linear filter,
sharpening,
an artifact removal mapping,
deconvolution,
histogram spreading,
down-sampling, and
inpainting of the microscope image, wherein the transforming is carried out in particular using a trained processing model.

25. An evaluation device for evaluating microscope images, comprising means for carrying out the method according to claim 1.

26. An image processing system comprising an evaluation device according to the preceding claim 25, in particular comprising an imaging device such as a microscope.

27. A machine learning system for training a processing model according to claim 1.

28. A computer program product comprising instructions which, when the program is executed by a computer, cause the latter to carry out the method according to claim 1, the computer program product being in particular a computer-readable storage medium.

29. An image processing system comprising an evaluation device, wherein the evaluation device comprises a processing model which has been trained according to the method according to claim 1 to carry out a virtual processing mapping, in particular comprising an imaging device such as a microscope, wherein the evaluation device is in particular designed to process the images recorded with the imaging device by the learned virtual processing mapping.

30. Method for generating a resulting microscope image with a machine learning system having a processing model for microscope images of samples of a sample type, comprising:
providing of a processing model for carrying out a virtual processing mapping for microscope images of the sample type, wherein a processing model is used which has been trained using a method for training a machine learning system according to claim 1,
recording a coarse stack to be processed comprising at least one or more microscope images of the sample of the sample type, the microscope images registered to one another,
calculating a resulting microscope image from the coarse stack to be processed using the virtual processing mapping,
characterized in that the coarse stack to be processed resolves the sample coarser than the fine stack, in particular the number of microscope images in the coarse stack to be processed is smaller than the number of microscope images in the fine stack.

31. The method according to the preceding claim 30, wherein the method further comprises, before the providing of the processing model:
verifying whether a suitable processing model having a suitable processing mapping for the sample of the sample type is available and, if not, executing the method for training a machine learning system according to claim 1 using the sample, wherein in particular the fine stack is created in a predetermined region of the sample and in particular the coarse stack to be processed is recorded in a region of the sample different from the predetermined region.

32. The method according to claim 31, wherein the verifying whether a suitable processing model is available comprises:
selecting a processing model, and
verifying a quality of the resulting microscope image, and if the quality of the resulting microscope image does not meet a minimum requirement, executing the method for training a machine learning system, wherein the verifying a quality in particular comprises one or more of:
a manual verifying,
a matching with example target images,
a metric in particular based on edge sharpness, artefacts, expected structures, wherein artefacts and expected structures in particular can be identified using a metric quality model, and the metric quality model has been trained to identify the artefacts and the expected structures,
inputting into a quality classification model that has been trained to identify well and poorly reconstructed microscope images,
verifying on the basis of image features such as image sharpness, noise level, blood flow, artefacts such as ringing and striping artefacts.

33. The method according to claim 30, wherein the number of microscope images in the coarse stack to be processed is equal to the number of microscope images in the coarse stack of the annotated data set, and in particular both the coarse stack to be processed and the coarse stack of the annotated data set comprise a plurality of microscope images.
